(19) United States
(12) Patent Application Publication
(10) Pub. No.: US 2025/0009654 A1
(43) Pub. Date: Jan. 9, 2025

(54) STRUCTURED RHEOLOGICAL SOLID PERSONAL CARE COMPOSITION

(71) Applicant: The Procter & Gamble Company, Cincinnati, OH (US)

(72) Inventors: Matthew Lawrence LYNCH, Mariemont, OH (US); Brandon Philip ILLIE, Felicity, OH (US); Taotao ZHU, West Chester, OH (US); Jamie Lynn DRIA, Deerfield Township, OH (US); Tinlee LIN, Cincinnati, OH (US); Baltej LUDHER, Cincinnati, OH (US); Greg VEINTIMILLA, Maineville, OH (US); Corrie MOLL, Cincinnati, OH (US)

(21) Appl. No.: 18/888,534

(22) Filed: Sep. 18, 2024

Related U.S. Application Data
(63) Continuation of application No. 17/225,218, filed on Apr. 8, 2021.
(60) Provisional application No. 63/081,436, filed on Sep. 22, 2020.
(60) Provisional application No. 63/007,963, filed on Apr. 10, 2020.

(51) Int. Cl. A61K 9/06; A61K 8/02; A61K 8/34; A61K 8/36; A61K 8/73; A61K 8/92; A61K 8/9767; A61K 31/05; A61K 36/15; A61K 36/61; A61K 47/10; A61K 47/36; A61K 47/44; A61Q 90/00

(52) U.S. Cl. CPC A61K 9/06; A61K 8/0229; A61K 8/345; A61K 8/347; A61K 8/361; A61K 8/73; A61K 8/922; A61K 8/9767; A61K 31/05; A61K 36/15; A61K 36/61; A61K 47/10; A61K 47/36; A61K 47/44; A61Q 90/00; A61K 2800/28; A61K 2800/524; A61K 2800/59; A61K 2800/805

(57) ABSTRACT

A rheological solid personal care composition comprises a crystallizing agent, a suspension agent, an insoluble active, and an aqueous phase. A process for the manufacture of a rheological solid composition comprises the steps of: provision and heat up of an aqueous solution of sodium chloride, and sodium hydroxide; addition of an emulsifier, preferably palmitic acid, in order to obtain an emulsifier main mix, preferably a sodium palmitate soap main mix; addition of a suspension agent, preferably xanthan gum and glycerin, to the emulsifier main mix; addition of an insoluble active premix to the emulsifier main mix to obtain a blend, the insoluble active premix preferably being a petrolatum-based premix of topical drug actives, the topical drug active preferably selected from the group of: menthol, nutmeg, camphor, eucalyptus, cedar leaf, thymol, and any combinations thereof; cool down of the blend in order to form a crystalline structure of the rheological solid composition; optionally, addition of a hygroscopic stabilizer to the blend in order to stabilize the crystalline structure, the hygroscopic stabilizer preferably being sodium lactate.

### FIELD OF THE INVENTION

Described herein is a rheological solid liquid expressing personal care composition comprising more than about 55% water having a crystallizing agent with an elongated, fiber-like crystal habit. The rheological solid personal care composition may allow for a unique skin feel “crunch” and/or glide when rubbed on the skin, and may provide an enhanced evaporative cooling for a refreshing/cooling sensation, even in the absence of sensate.

### BACKGROUND OF THE INVENTION

Personal care compositions are routinely used by consumers on the chest, back, and/or throat to provide relief from nasal congestion, dry cough, chest congestion, muscle aches and/or pains, difficulty sleeping due to the common cold and/or flu, and/or provide a soothing feeling on the skin. Current products are formulated as creams, lotions, and/or ointments and are applied to the skin by hand, which can be messy and hard to control where the product is applied due to their liquid or semi-liquid properties. Such products can also leave a greasy feeling on the user's hands after application and/or may leave stains on clothing and sheets. In addition, some consumers may desire to apply such products multiple times throughout the day or while on-the-go without having to wash their hands after application. As such, there is a need for a more convenient, non-messy delivery system for personal care compositions.

Conventional soap-type gel-sticks are commonly used as deodorant for underarm application, and typically incorporate sodium stearate (C18) gelling agents (which are really a mixture of chain lengths derived from the natural source of stearate-typically tallow). The use of sodium stearate requires the inclusion of high levels of polyols (e.g. propylene glycol and glycerin) as a solubility aid for the gelling agent during processing, even at high process temperatures. Typical compositions include about 50% propylene glycol, 25% glycerin and only 25% water (EP2170257 and EP2465487). This eliminates the crunch and mutes the glide feel and cooling sensation of the solid stick. Finally, this may require high levels of gelling agent, including gelling agents other than sodium stearate, to produce gel-sticks and particularly translucent gel-sticks.

Attempts have been made to provide rheological solid compositions similar in composition to those embodied in this invention, comprising insoluble active agents such as perfume capsules, solid particles, or oil droplets because rheological solid compositions provide a way for a user to quickly and easily apply a rheological solid composition to a particular surface. However, these products do not stabilize the insoluble active agents in the compositions, resulting in the insoluble active agents either floating to the top (i.e. ‘creaming’) or settling to the bottom (i.e. ‘sedimenting’) before the composition solidifies. If the insoluble active agents are not evenly distributed, a rheological solid composition may have a higher insoluble active agent concentration in one region versus another, resulting in uneven performance during the lifetime use of the product. In the most egregious cases, it is unacceptable from a consumer product to have noticeable amounts of insoluble actives on the top and/or bottom of the product; most preferred is to have insoluble active evenly dispersed throughout the product.

There is a need to deliver a rheological solid personal care composition having low levels of gelling agent that can retain its shape and comprises insoluble active benefit agents that are uniformly suspended in the composition.

### SUMMARY OF THE INVENTION

A rheological solid personal care composition is provided that comprises crystallizing agent; suspension agent; insoluble active; and an aqueous phase.

Further, a rheological solid composition for use in a method of treating: nasal congestion, common cold, flue, cough, dry cough, chest congestion, muscle aches and pains, or any combinations thereof, is provided.

Further, a process for the manufacture of a rheological solid composition is provided, the process comprising the following steps:
provision and heat up of an aqueous solution of sodium chloride, and sodium hydroxide,
addition of an emulsifier, preferably palmitic acid, in order to obtain an emulsifier main mix, preferably a sodium palmitate soap main mix,
addition of a suspension agent, preferably xanthan gum and glycerin, to the emulsifier main mix,
addition of an insoluble active premix to the emulsifier main mix to obtain a blend, the insoluble active premix preferably being a petrolatum-based premix of topical drug actives, the topical drug active preferably selected from the group of: menthol, nutmeg, camphor, eucalyptus, cedar leaf, thymol, and any combinations thereof,
cool down of the blend in order to form a crystalline structure of the rheological solid composition,
optionally addition of a hygroscopic stabilizer to the blend in order to stabilize the crystalline structure, the hygroscopic stabilizer preferably being sodium lactate.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1A. Top view showing separation of actives, in the absence of suspension agent(s).

FIG. 1B. Side view showing separation of actives, in the absence of suspension agent(s).

FIG. 2A. Top view showing NO separation of actives, in the presence of suspension agent(s).

FIG. 2B. Side view showing NO separation of actives, in the presence of suspension agent(s).

FIG. 3. SEM of crystalline mesh formed of fiber-like particles.

FIG. 4. Effective Gum Suspension Agent Systems for Stabilization of Insoluble Active Particles.

FIG. 5. Effect of Gum Suspension Agents on the Effectiveness of Different Crystallizing Agents.

FIG. 6. Total Fragrance Expression (Concentration in ppm) vs. Time (Hours).

### DETAILED DESCRIPTION OF THE INVENTION

The present invention includes a rheological solid personal care composition comprising a crystalline mesh. The crystalline mesh (“mesh”) comprises a relatively rigid, three-dimensional, interlocking crystalline skeleton frame of fiber-like crystalline particles (formed from crystallizing agents), having voids or openings containing aqueous solution and optionally one or more actives. The mesh provides a self-supporting structure, such that a rheological solid personal care composition may ‘stand on its own’ when resting on a surface. If compressed above a critical stress, the mesh allows the rheological solid personal care composition to express the entrapped aqueous solution, and optionally one or more actives. The rheological solid personal care compositions of the present invention include crystallizing agent(s), suspension agent(s), insoluble active(s), and aqueous phase, and may be combined with a device to enable application.

As used herein, “personal care composition” refers to compositions intended for topical application to the skin, including topical prescription medications, over-the-counter medications, behind-the-counter medications, cosmetics, consumer goods, and combinations thereof.

### Crystallizing Agent(s)

In the present invention, the mesh of a rheological solid personal care composition includes fiber-like crystalline particles formed from crystallizing agents; wherein “crystallizing agent” as used herein includes sodium salts of fatty acid with shorter chain length (C12-C20), such as sodium palmitate (C16) in majority of water. The rheological solid personal care compositions are best achieved with a ‘narrow’ distribution of crystallizing agent chain lengths, further best achieved in the absence of very short chain lengths (C12 or shorter) and measurable amounts of unsaturation on the chains of the fatty acid sodium salts, coupled with controlled crystallization processing. One skilled in the art recognizes crystalline particles as exhibiting sharp scattering peaks between 0.25 −60 deg. 2θ in powdered x-ray diffraction measurements. This is in sharp contrast to compositions in which these materials are used as gelling agents, which show broad amorphic scattering peaks emanating from poorly formed solids.

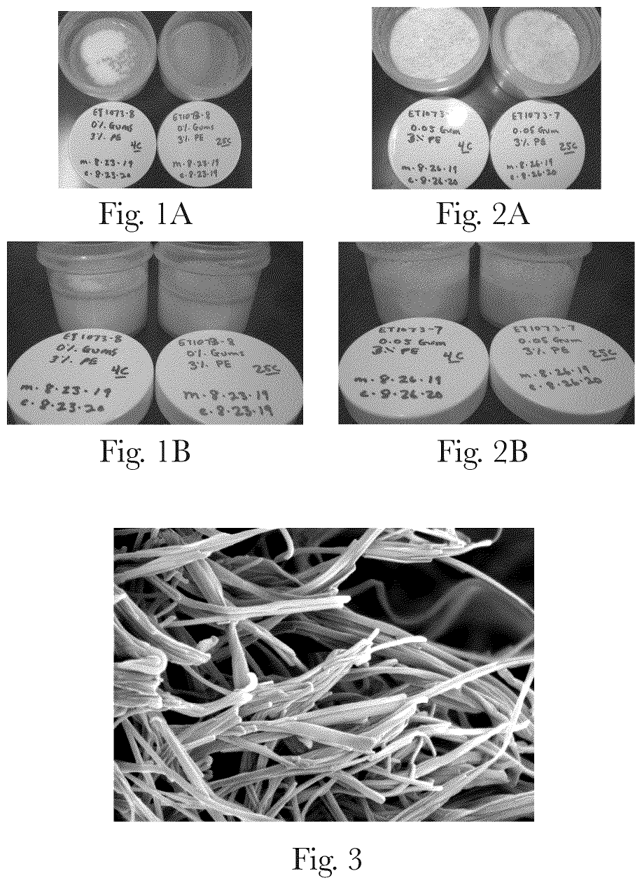

Rheological solid personal care compositions can comprise greater than about 55% water, alternatively greater than about 60%, alternatively greater than about 65%, alternatively greater than about 70% water, alternatively greater than about 80% water, and are ‘structured’ by a mesh of interlocking, fiber-like crystalline particles of mostly single-chain length, as described above (see FIG. 3). The term “fiber-like crystalline particle” refers to a particle in which the length of the particle in the direction of its longest axis is greater than 10× the length of the particle in any orthogonal direction. The fiber-like crystalline particles produce a mesh at very low concentrations (˜0.5 wt %) that create a solid that yields only with a minimum applied stress-i.e. rheological solid. The suspension agent(s), insoluble active(s) and aqueous phase (water) primarily reside in the open spaces of the mesh. In preparing these compositions, the crystallizing agent is dissolved in water using heat. The fiber-like crystalline particles form into the mesh as the mixture cools over minutes to hours. Not wishing to be bound by theory, but the suspension agents-such as polymer gums, clay particles and hydrophobic fat particles, prevent the insoluble actives from creaming or sedimenting during the formation of the mesh (See FIGS. 1A and 1B); the removal of the suspension agents show significant (or catastrophic) separation of the insoluble active(s). Preferred compositions have a phase stability grade of ‘1’ and most preferred phase stability grade of ‘2’, as determined by the PHASE STABILITY TEST METHOD, described herein.

Without being limited to theory, it is thought that only sodium salts of fatty acid with high chain length can function as crystallizing agents in the present invention. The inclusion of shorter chain length (C12 or shorter) crystallizing agents can make the compositions too soluble at room temperature, such that the fiber-like crystalline particles do not form. The inclusion of unsaturation in chains of the sodium salts of fatty acid adds too many ‘kinks’ for crystallization, such that the fiber-like crystalline particles do not form and the compositions are mush or liquid. The crystallizing agent should be present in sufficient quantity to create a rheological solid with a firmness between about 0.1 N and about 50.0 N, more preferably between about 0.5 N—about 40.0 N, more preferably between about 1.0 N—about 30.0 N and most preferably between about 2.5 N —about 15.0 N, where the lower value sets a minimum ‘softness’ to the composition and the upper value sets a maximum ‘hardness’ to the composition, both of which are influenced by the consumer product application. In some aspects, the crystallizing agent is present in an amount from about 0.01% to about 10%. by weight of the rheological solid personal care composition. The crystallizing agent may be present in an amount of from about 0.1% to about 7%, by weight of the rheological solid personal care composition, from about 1% to about 7%, by weight of the rheological solid personal care composition, or from about 2% to about 5%, by weight of the rheological solid personal care composition.

The crystallizing agent should form elongate fiber-like crystalline particles, in which the length of the particle in the direction of its longest axis is preferably greater than 10× the length of the particle in any orthogonal direction, more preferably greater than 15×, and most preferably greater than 20×, as assessed by standard Scanning Electron Microscopy (SEM) methods. Not wishing to be bound by theory, but longer crystalline particles are thought to intertwine more efficiently creating efficient mesh structures. This contrasts with fatty acid crystals (protonated version of the sodium salt of fatty acid) of magnesium salt of fatty acid which are not-elongated and generally exhibit a ratio of 1× to 2×. The composition of the fiber-like crystalline particles should be thermally stable at room temperature, with preferred temperatures greater than about 30° C., more preferably greater than about 35° C., more preferably greater than about 40° C., more preferably greater than about 50° C., most preferably greater than about 60° C., as determined by the THERMAL STABILITY TEST METHOD, as described herein. Finally, the fiber-like crystalline particles combine to form a mesh, such that the aqueous phase and insoluble actives can be expressed from the rheological solid personal care composition with a defined applied stress. The work required to express aqueous phase from 15% of the volume of the structure of the rheological solid personal care composition is preferably between about 100 J m-3 and about 6000 J m-3, alternatively from about 100 J m-3 and about 3000 J m-3, alternatively between about 300 J m-3 and about 2000 J m-3, alternatively between about 500 J m-3 and about 1500 J m-3, as determined by the WATER-EXPRESSION TEST METHOD, as described herein.

In some aspects, the crystallizing agent can be a metal salt. Non-limiting examples of metals salts can include sodium stearate, sodium palmitate, potassium stearate, potassium palmitate, sodium myristate. One of skill in the art would understand that the rheological solid personal care composition can be made using the acid form of the salt in combination with a base, such as sodium hydroxide, to form the metal salt.

### Suspension Agent(s)

The suspension agent prevents the separation of insoluble actives in the preparation of the rheological solid personal care composition. Inventive compositions are heated until the crystallizing agent is dissolved leaving a dispersed active in a low viscosity fluid. When the compositions are cooled, the crystallizing agent begins to form fiber-like crystalline particles which weave together into the mesh, which eventually traps the actives. This process can take minutes to hours. Not wishing to be bound by theory, it is believed that the suspension agents increase viscosity or create a yield stress that holds the actives from creaming or sedimenting during the crystallization of the crystallizing agent and formation of the mesh. Preferred suspension agents are effective at low concentrations to prevent potential negative effects on the mesh and performance of the consumer product. Preferred levels are below about 2 wt. %, alternatively below about 1 wt. %, alternatively below about 0.5 wt. %, alternatively below about 0.1 wt. %. In some aspects, the rheological solid personal care composition can comprise from about 0.01 to about 2 wt. % of a suspension agent, alternatively from about 0.05 to about 1 wt. %, alternatively from about 0.1 to about 0.5 wt. %, alternatively from about 0.25 to about 0.35 wt. %, all by weight of the rheological solid personal care composition.

Suitable suspension agents include gums, polymers, microfiber particles, clay particles, and combinations thereof, and unexpectedly must be selected for a composition such that their addition does not have a negative effect on the mesh. For example, the use of gums can weaken the mesh structure relative to compositions that do not contain gums, requiring an increase in the amount of crystallizing agent (Example 2). As another example, use of clays (Example 10) and microfibers (Example 9) can be rendered ineffective with the addition of sodium chloride.

### Gums

The rheological solid personal care composition includes at least one suspension agent to keep insoluble materials (i.e. solids or oils) suspended during preparation. The suspension agent may include one or more biopolymers. Non-limiting examples of such biopolymers include polysaccharides such as polymers of glucose, fructose, galactose, mannose, rhamnose, glucuronic acid, and mixtures thereof.

The suspension agent may be in the form of a polysaccharide or mixture of polysaccharides. Preferable polysaccharide suspension agents include xanthan gum, glucomannan, galactomannan, and combinations thereof. The glucomannan may be derived from a natural gum such as konjac gum. The galactomannan may be derived from natural gums such as locust bean gum. Polysaccharide suspension agents may also include carrageenan. Suspension agent gums may be modified such as by deacetylation.

The rheological solid personal care composition may comprise a polysaccharide suspension agent system comprising at least two polysaccharides, such as a first polysaccharide and a second polysaccharide. The first polysaccharide may be xanthan gum. The second polysaccharide may be selected from the group consisting of glucomannan, galactomannan, and combinations thereof. The second polysaccharide may be selected from the group consisting of konjac gum, locust bean gum, tara bean, and combinations thereof.

Preferably, the first polysaccharide is xanthan gum and the second polysaccharide is konjac gum.

The first polysaccharide may be present at a level of greater than about 10 wt. % and less than about 100 wt. %, alternatively about 40 wt. % to about 90 wt. %, alternatively about 40 wt. % to about 60 wt. %, by weight of the polysaccharide suspension agent system.

The second polysaccharide may be present at a level of about 0 wt. % to about 90 wt. %, alternatively about 60 wt. % to about 10 wt. %, alternatively about 60 wt. % to about 40 wt. %, by weight of the polysaccharide suspension agent system.

The total concentration of polysaccharide present in the rheological solid personal care composition may be between about 0.01—about 1.0 wt. %, or more preferably between about 0.03 —about 1.0 wt. %, or more preferably between about 0.05—about 0.8 wt. %, more preferably between about 0.07—about 0.75 wt. %, and most preferably between about 0.09—about 0.5 wt. %, all by weight of the rheological solid personal care composition. Without wishing to be bound by theory, it is believed that minimizing the total polysaccharide level in the composition ensures stability of the dispersed active agents during preparation while minimizing the effect of the suspension agent on the mesh structure.

The polysaccharide suspension agent system may have a weight-average molecular weight in the range of about 10,000 Daltons to about 15,000,000 Daltons, alternatively about 200,000 Daltons to about 10,000,000 Daltons, alternatively about 300,000 Daltons to about 6,000,000 Daltons, alternatively about 300,000 Daltons to about 500,000 Daltons.

The polysaccharide suspension agent system may be characterized by the average ratio of acetylation, wherein the average ratio of acetylation is the number of acetylated hydroxyl groups in the polysaccharide divided by the number of free hydroxyl groups in the polysaccharide. The average ratio of acetylation may be in the range of about 2.0 to about 0.5, preferably in the range of about 1.5 to about 0.5.

### Clays

In the present disclosure, a suspension agent may be used to provide viscosity and thixotropic properties to the composition so that the suspended active agent particles are prevented from creaming or settling during preparation. In one or more embodiments, the suspension agent may be a mineral clay mixture and more particularly an organophilic mineral clay mixture. In one or more embodiments, the mineral clay mixture may be treated with alkyl quaternary ammonium compounds in order to render the mineral clay mixture hydrophobic; such clays may also be termed organophilic. In one or more embodiments, the mineral clay mixtures can comprise: a mineral clay (a) comprising from about 50 to about 95 wt. %, based on the weight of the mineral clay mixture, or from about 60 to about 95 wt. %, or from about 70 to about 90 wt. %, of a mineral clay selected from the group consisting of sepiolite, palygorskite, and mixtures of sepiolite and palygorskite; and a mineral clay (b) comprising the balance, by weight of the mineral clay mixture, of a smectite. In one or more embodiments, the smectite may be a natural or synthetic clay mineral selected from the group consisting of hectorite, laponite, montmorillonite, bentonite, beidelite, saponite, stevensite, and mixtures thereof. Suitable clays include Laponite from the Garamite line of products available from BYK Additives, (Gonzalez, TX).

### Microfibers

Any microcrystalline cellulose may be employed in the compositions of the present invention. Suitable feedstocks include, for example, wood pulp such as bleached sulfite and sulfate pulps, corn husks, bagasse, straw, cotton, cotton linters, flax, kemp, ramie, fermented cellulose, etc. The amounts of microcrystalline cellulose and hydrocolloid may be varied over a wide range depending upon the properties desired in the final composition. Suitable microfibers include Rheocrysta c-2sp (WASE COSFA USA, Inc.).

### Insoluble Active(s)

The rheological solid personal care composition may include one or more insoluble active particles besides the fiber-like crystalline particles that comprise the mesh. As used herein, an “insoluble active particle” comprises at least a portion of a solid, a semi-solid, or liquid material, including some amount of insoluble active. The insoluble active particles may take various different forms, for example the insoluble active particles may be 100 wt. % solid or may be hollow. The insoluble active particles may include, for example, mesoporous particles, activated carbon, zeolites, benefit agent delivery particles, waxes, insoluble oils, hydrogels, and/or ground nutshells.

In some aspects, the rheological solid personal care composition can comprise from about 0.001 to about 35 wt. % insoluble active particles, alternatively from about 0.01 to about 30 wt. % insoluble active particles, alternatively from about 0.01 to about 25% insoluble active particles, alternatively from about 0.1 to about 15 wt. %, alternatively from about 0.5 to about 12 wt. %, alternatively from about 1 to about 10 wt. %, alternatively from about 5 to about 10 wt. %, all by weight of the rheological solid personal care composition.

In some aspects, the rheological solid personal care composition can comprise from about 0.001 to about 30 wt. % insoluble active, alternatively from about 0.1 to about 30 wt. %, alternatively from about 0.1 to about 25 wt. %, alternatively from about 0.5 to about 15 wt. %, alternatively from about 1 to about 10 wt. %, alternatively from about 5 to about 15 wt. %, all by weight of the rheological solid personal care composition.

The rheological solid personal care composition may include one or more types of insoluble active particles, for example, two insoluble active particles types, wherein one of the first or second insoluble active particles (a) is made of a different material than the other; (b) has a wall that includes a different amount of wall material or monomer than the other; (c) contains a different amount of perfume oil ingredient than the other; (d) contains a different perfume oil; (c) has a wall that is cured at a different temperature; (f) contains a perfume oil having a different cLogP value; (g) contains a perfume oil having a different volatility; (h) contains a perfume oil having a different boiling point; (i) has a wall made with a different weight ratio of wall materials; (j) has a wall that is cured for different cure time; and/or (k) has a wall that is heated at a different rate.

The plurality of insoluble active agent particles may have a diameter of less than about 500 μm, alternatively less than about 400 μm, alternatively less than about 300 μm, alternatively less than about 200 μm, alternatively less than about 100 μm. One skilled in the art recognizes that the ability to suspend particles is a function of the mean diameter of the particles (where larger particles are more difficult to suspend) and a function of the total amount of the particles (where large amounts of particles are more difficult to suspend).

To the former, one skilled in the art further recognizes that the concentration of the suspension agent with a given insoluble active agent may have to be increased to accommodate larger insoluble active particles. It is generally preferred to minimize the amount of suspension agent (e.g. Example 2) so that smaller active agent particles are preferred. To the latter, one skilled in the art further recognizes that the concentration of the suspension agent with a given insoluble active agent may have to be increased to accommodate larger amounts of insoluble active particles (e.g. Example 7).

### Encapsulated Insoluble Benefit Agent

The insoluble active particle may include a wall material that encapsulates an insoluble active. The insoluble active may be selected from the group consisting of: perfume compositions, perfume raw materials, perfume, skin coolants, vitamins, sunscreens, antioxidants, glycerin, bleach encapsulates, chelating agents, antistatic agents, insect and moth repelling agents, colorants, antioxidants, sanitization agents, disinfecting agents, germ control agents, mold control agents, mildew control agents, antiviral agents, drying agents, stain resistance agents, soil release agents, chlorine bleach odor control agents, dye fixatives, dye transfer inhibitors, color maintenance agents, optical brighteners, color restoration/rejuvenation agents, anti-fading agents, whiteness enhancers, anti-abrasion agents, wear resistance agents, fabric integrity agents, anti-wear agents, anti-pilling agents, defoamers, anti-foaming agents, UV protection agents, sun fade inhibitors, anti-allergenic agents, enzymes, water proofing agents, fabric comfort agents, shrinkage resistance agents, stretch resistance agents, stretch recovery agents, skin care agents, natural actives, antibacterial actives, antiperspirant actives, cationic polymers, dyes, metal catalysts, non-metal catalysts, activators, pre-formed peroxy-carboxylic acids, diacyl peroxides, hydrogen peroxide sources, enzymes, topical drug actives, and combinations thereof. As used herein, a “perfume raw material” refers to one or more of the following ingredients: fragrant essential oils; aroma compounds; pro-perfumes; materials supplied with the fragrant essential oils, aroma compounds, and/or pro-perfumes, including stabilizers, diluents, processing agents, and contaminants; and any material that commonly accompanies fragrant essential oils, aroma compounds, and/or pro-perfumes.

The wall material of the insoluble active particle may comprise melamine, polyacrylamide, silicones, silica, polystyrene, polyurea, polyurethanes, polyacrylate based materials, polyacrylate ester-based materials, gelatine, styrene malic anhydride, polyamides, aromatic alcohols, polyvinyl alcohol and mixtures thereof. The melamine wall material may comprise melamine crosslinked with formaldehyde, melamine-dimethoxyethanol crosslinked with formaldehyde, and mixtures thereof. The polystyrene wall material may comprise polystyrene cross-linked with divinylbenzene. The polyurea wall material may comprise urea crosslinked with formaldehyde, urea crosslinked with gluteraldehyde, polyisocyanate reacted with a polyamine, a polyamine reacted with an aldehyde and mixtures thereof. The polyacrylate based wall materials may comprise polyacrylate formed from methylmethacrylate/dimethylaminomethyl methacrylate, polyacrylate formed from amine acrylate and/or methacrylate and strong acid, polyacrylate formed from carboxylic acid acrylate and/or methacrylate monomer and strong base, polyacrylate formed from an amine acrylate and/or methacrylate monomer and a carboxylic acid acrylate and/or carboxylic acid methacrylate monomer, and mixtures thereof.

The polyacrylate ester-based wall materials may comprise polyacrylate esters formed by alkyl and/or glycidyl esters of acrylic acid and/or methacrylic acid, acrylic acid esters and/or methacrylic acid esters which carry hydroxyl and/or carboxy groups, and allylgluconamide, and mixtures thereof.

The aromatic alcohol-based wall material may comprise aryloxyalkanols, arylalkanols and oligoalkanolarylethers. It may also comprise aromatic compounds with at least one free hydroxyl-group, especially preferred at least two free hydroxy groups that are directly aromatically coupled, wherein it is especially preferred if at least two free hydroxy-groups are coupled directly to an aromatic ring, and more especially preferred, positioned relative to each other in meta position. It is preferred that the aromatic alcohols are selected from phenols, cresols (o-, m-, and p-cresol), naphthols (alpha and beta-naphthol) and thymol, as well as ethylphenols, propylphenols, fluorphenols and methoxyphenols.

The polyurea based wall material may comprise a polyisocyanate. The polyisocyanate may be an aromatic polyisocyanate containing a phenyl, a toluoyl, a xylyl, a naphthyl or a diphenyl moicty (e.g., a polyisocyanurate of toluene diisocyanate, a trimethylol propane-adduct of toluene diisocyanate or a trimethylol propane-adduct of xylylene diisocyanate), an aliphatic polyisocyanate (e.g., a trimer of hexamethylene diisocyanate, a trimer of isophorone diisocyanate and a biuret of hexamethylene diisocyanate), or a mixture thereof (e.g., a mixture of a biuret of hexamethylene diisocyanate and a trimethylol propane-adduct of xylylene diisocyanate). In still other embodiments, the polyisocyanate may be cross-linked, the cross-linking agent being a polyamine (e.g., diethylenetriamine, bis(3-aminopropyl)amine, bis(hexanethylene)triamine, tris(2-aminoethyl)amine, tricthylenetetramine, N,N′-bis(3-aminopropyl)-1,3-propanediamine, tetraethylenepentamine, pentaethylenchexamine, branched polyethylenimine, chitosan, nisin, gelatin, 1,3-diaminoguanidine monohydrochloride, 1,1-dimethylbiguanide hydrochloride, or guanidine carbonate).

The polyvinyl alcohol based wall material may comprise a crosslinked, hydrophobically modified polyvinyl alcohol, which comprises a crosslinking agent comprising i) a first dextran aldehyde having a molecular weight of from about 2,000 to about 50,000 Da; and ii) a second dextran aldehyde having a molecular weight of from greater than about 50,000 to about 2,000,000 Da.

Preferably, the insoluble active particle with perfume has a wall material comprising silica or a polymer of acrylic acid or derivatives thereof and a benefit agent comprising a perfume mixture.

With regards to insoluble active particles, the rheological solid personal care composition may contain from about 0.001 wt. % to about 20 wt. %, by weight of the rheological solid personal care composition, of a benefit agent contained with the wall material of the benefit agent delivery particle. Or, the rheological solid personal care composition may contain from about 0.01 wt. % to about 10 wt. %, or most preferably from about 0.05 wt. % to about 5 wt. %, by weight of the rheological solid personal care composition, of a benefit agent contained with the wall material of the insoluble active particle.

These walled particles may be coated with a deposition aid, a cationic polymer, a non-ionic polymer, an anionic polymer, or mixtures thereof. Suitable polymers may be selected from the group consisting of: polyvinylformaldehyde, partially hydroxylated polyvinylformaldehyde, polyvinylamine, polyethyleneimine, ethoxylated polyethyleneimine, polyvinylalcohol, polyacrylates, and combinations thereof.

### Unencapsulated Perfume

The rheological solid personal care composition may include unencapsulated perfume comprising one or more perfume raw materials that solely provide a hedonic benefit (i.e. that do not neutralize malodors yet provide a pleasant fragrance). Suitable perfumes are disclosed in U.S. Pat. No. 6,248,135. For example, the rheological solid personal care composition may include a mixture of volatile aldehydes for neutralizing a malodor and hedonic perfume aldehydes.

Where perfumes, other than the volatile aldehydes in the malodor control component, are formulated into the rheological solid personal care composition, the total amount of perfumes and volatile aldehydes may be from about 0.015 wt. % to about 2 wt. %, alternatively from about 0.01wt. % to about 1.0 wt. %, alternatively from about 0.015 wt. % to about 0.5 wt. %, by weight of the rheological solid personal care composition.

### Perfume Delivery Technologies

The rheological solid personal care compositions may comprise one or more perfume delivery technologies that stabilize and enhance the deposition and release of perfume ingredients from a treated substrate. Such perfume delivery technologies can also be used to increase the longevity of perfume release from the treated substrate. Perfume delivery technologies, methods of making certain perfume delivery technologies and the uses of such perfume delivery technologies are disclosed in US 2007/0275866 A1.

The rheological solid personal care compositions may comprise from about 0.001 wt. % to about 20 wt. %, or from about 0.01 wt. % to about 10 wt. %, or from about 0.05 wt. % to about 5 wt. %, or even from about 0.1 wt. % to about 0.5 wt. %, by weight of the perfume delivery technology. In one aspect, the perfume delivery technologies may be selected from the group consisting of: pro-perfumes, polymer particles, soluble silicone, polymer assisted delivery, molecule assisted delivery, assisted delivery, amine assisted delivery, cyclodextrins, starch encapsulated accord, zeolite and inorganic carrier, and mixtures thereof.

The perfume delivery technology may comprise an amine reaction product (ARP) or a thio reaction product. One may also use “reactive” polymeric amines and or polymeric thiols in which the amine and/or thiol functionality is pre-reacted with one or more PRMs to form a reaction product. Typically, the reactive amines are primary and/or secondary amines, and may be part of a polymer or a monomer (non-polymer). Such ARPs may also be mixed with additional PRMs to provide benefits of polymer-assisted delivery and/or amine-assisted delivery. Nonlimiting examples of polymeric amines include polymers based on polyalkylimines, such as polyethyleneimine (PEI), or polyvinylamine (PVAm). Nonlimiting examples of monomeric (non-polymeric) amines include hydroxyl amines, such as 2-aminoethanol and its alkyl substituted derivatives, and aromatic amines such as anthranilates. The ARPs may be premixed with perfume or added separately in leave-on or rinse-off applications. In another aspect, a material that contains a heteroatom other than nitrogen and/or sulfur, for example oxygen, phosphorus or selenium, may be used as an alternative to amine compounds. In yet another aspect, the aforementioned alternative compounds can be used in combination with amine compounds. In yet another aspect, a single molecule may comprise an amine moiety and one or more of the alternative heteroatom moieties, for example, thiols, phosphines and selenols. The benefit may include improved delivery of perfume as well as controlled perfume release. Suitable ARPs as well as methods of making same can be found in USPA 2005/0003980 A1 and USP 6,413,920 B1.

### Essential and Natural Oils

The insoluble active particle may include individual mixtures of insoluble oils such as essential and natural oils. The term “essential oils” as used herein refers to oils or extracts distilled or expressed from plants and constituents of these oils. Typical essential oils and their main constituents are those obtained for example from thyme (thymol, carvacrol), oregano (carvacrol, terpenes), lemon (limonene, terpinene, phellandrene, pinene, citral), lemongrass (citral, methylheptenone, citronellal, geraniol), orange flower (linalool, β-pinene, limonene), orange (limonene, citral), anise (anethole, safrol), clove (eugenol, eugenyl acetate, caryophyllene), rose (geraniol, citronellol), rosemary (borneol, bornyl esters, camphor), geranium (geraniol, citronellol, linalool), lavender (linalyl acetate, linalool), citronella (geraniol, citronellol, citronellal, camphene), eucalyptus (cucalyptol); peppermint (menthol, menthyl esters), spearmint (carvone, limonene, pinene), wintergreen (methyl salicylate), camphor (safrole, acetaldehyde, camphor), bay (eugenol, myrcene, chavicol), cinnamon (cinnamaldehyde, cinnamyl acetate, eugenol), tea tree (terpinen-4-ol, cincole), eucalyptus oil, nutmeg oil, turpentine oil, chamomile oil, neroli oil, cedar leaf (α-thujone, β-thujone, fenchone), and combinations thereof. Essential oils are widely used in perfumery and as flavorings, medicine, and solvents. Essential oils, their composition and production, are described in detail in Kirk-Othmer Encyclopedia of Chemical Technology, 4^{th} Edition and in The Merck Index, 13^{th} Edition.

In some aspects, the rheological solid personal care composition can comprise from about 0.1 to about 20 wt. % insoluble oils, alternatively from about 0.5 to about 15 wt. %, alternatively from about 1 to about 12 wt. %, alternatively from about 4 to about 15 wt. %, alternatively from about 5 to about 10 wt. %, all by weight of the rheological solid personal care composition.

### Waxes and Oils

The insoluble active particle may include individual mixtures of waxes and oils as a non-aqueous vehicle. The non-aqueous vehicle is generally any chemical in any physical form that does not contain water. The non-aqueous vehicle can be selected from the group consisting of liquid petrolatum, petrolatum, mineral oil, glycerin, natural and synthetic oils, fats, silicone and silicone derivatives, polyvinylacetate, natural and synthetic waxes such as animal waxes like beeswax, lanolin and shellac, hydrocarbons, hydrocarbon derivatives, vegetable oil waxes such as carnauba, candelilla and bayberry wax, vegetable oils such as caprylic/capric triglycerides, and combinations thereof. In some aspects, the non-aqueous vehicle can be selected from the group consisting of liquid petrolatum, petrolatum, mineral oil, vegetable oils such as apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, and animal oil such as fish oil and oleic acid, and mixtures thereof. In some aspects, the non-aqueous vehicle can be mineral oil. In some aspects, the non-aqueous vehicle can be pentaerythrityl tetraisostearate.

Preferably, the non-aqueous vehicle is hydrophobic. One advantage to adding a hydrophobic non-aqueous vehicle, such as petrolatum, is thermal stability. Not wishing to be bound by theory, it is believed that the addition of a hydrophobic non-aqueous vehicle can provide better partitioning between the oil phase and aqueous phase, which can provide thermal stability. In addition, a hydrophobic non-aqueous vehicle can improve the hardness and spreadability of the rheological solid personal care composition.

In some aspects, the rheological solid personal care composition can comprise from about 1 to about 15 wt. % non-aqueous vehicle, alternatively from about 3 to about 12 wt. %, alternatively from about 5 to about 10 wt. %, all by weight of the rheological solid personal care composition.

In some aspects, the rheological solid personal care composition can comprise a ratio of insoluble active to non-aqueous vehicle of from about 1 to about 2, alternatively from about 1.5 to about 1.9.

### Malodor Counteractants

The rheological solid personal care composition may include other malodor reducing technologies. This may include, without limitation, amine functional polymers, metal ions, cyclodextrins, cyclodextrin derivatives, polyols, oxidizing agents, activated carbon, zeolites, and combinations thereof.

### Feel Modifiers

The rheological solid personal care composition may also include insoluble active agents designed to alter the feel properties of the composition when applied to surfaces, such as skin. This may include starches (e.g. tapioca starch, rice starch, or the like), talc, fumed silica (Aerosil® 200), titanium dioxide, dimethicone, iron oxide, mica, charcoal, colloidal oatmeal, colloidal cellulose, kaolin, and combinations thereof.

### Skin Care Agents

Skin care agents may be added to deliver a therapeutic and/or skin protective benefit. It will be recognized that of the numerous materials useful in the compositions delivered to skin, those that have been deemed safe and effective skin care agent and mixtures thereof are logical materials for use herein. Such materials include Category I actives as defined by the U.S. Food and Drug Administration's (FDA) Tentative Final Monograph on Skin Protectant Drug Products for Over-the-Counter Human Use (21 C.F.R. § 347), which presently include: allantoin, aluminum hydroxide gel, calamine, cocoa butter, dimethicone, cod liver oil (in combination), glycerine, kaolin, petrolatum, lanolin, mineral oil, shark liver oil, white petrolatum, tale, topical starch, zinc acetate, zinc carbonate, zinc oxide, and the like. Other potentially useful materials are Category DI actives as defined by the U.S. Food and Drug Administration's Tentative Final Monograph on Skin Protectant Drug Products for Over-the-Counter Human Use (21 C.F.R. § 347), which presently include: live yeast cell derivatives, aldioxa, aluminum acetate, microporous cellulose, cholecalciferol, colloidal oatmeal, cysteine hydrochloride, dexpanthenol, Peruvean balsam oil, protein hydrolysates, racemic methionine, sodium bicarbonate, Vitamin A, buffered mixture of cation and anion exchange resins, corn starch, trolamine, and the like. Further, other potential materials are Category II actives as defined by the U.S. Food and Drug Administration's Tentative Final Monograph on Skin Protectant Drug Products for Over-the-Counter Human Use (21 C.F.R. § 347), which include: bismuth subnitrate, boric acid, ferric chloride, polyvinyl pyrrolidone-vinyl acetate copolymers, sulfur, tannic acid, and the like. The skin care agent may be selected from these materials and mixtures thereof. As mentioned above, the materials for use should be safe. The rheological solid personal care composition may include between about 0.001 wt. % and about 20 wt. %, by weight of the rheological solid personal care composition, of the skin care agent. The concentration range of the skin care agents in the composition varies from material to material.

### Hair Treatment Actives

Pyridinethione anti-dandruff particulates, especially 1-hydroxy-2-pyridinethione salts, are suitable particulate anti-dandruff agents. The concentration of pyridinethione anti-dandruff particulate typically ranges from about 0.01 wt. % to about 5 wt. %, based on the total weight of the composition, generally from about 0.1 wt. % to about 3 wt. %, commonly from about 0.1 wt. % to about 2 wt. %. Suitable pyridinethione salts include those formed from heavy metals such as zinc, tin, cadmium, magnesium, aluminum and zirconium, generally zinc, typically the zinc salt of 1-hydroxy-2-pyridinethione (known as “zinc pyridinethione” or “ZPT”), commonly 1-hydroxy-2-pyridinethione salts in platelet particle form, wherein the particles have an average size of up to about 20 μm, typically up to about 5 μm, commonly up to about 2.5 μm. Salts formed from other cations, such as sodium, may also be suitable. Pyridinethione anti-dandruff agents are described, for example, in U.S. Pat. Nos. 2,809,971; 3,236,733; 3,753,196; 3,761,418; 4,345,080; 4,323,683; 4,379,753; and 4,470,982. As noted above, ZPT is a preferred pyridinethione salt.

In addition to the anti-dandruff active, compositions may also include one or more anti-fungal or anti-microbial actives in addition to the metal pyrithione salt actives. Suitable anti-microbial actives include coal tar, sulfur, charcoal, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Typical anti-microbials include itraconazole, ketoconazole, selenium sulphide and coal tar.

### Under Arm Treatment Actives

The rheological solid personal care composition may comprise from about 0.1 wt. % to about 50 wt. %, by weight of the rheological solid personal care composition, of a solubilized antiperspirant active suitable for application to human skin. The concentration of antiperspirant active in the composition should be sufficient to provide the finished antiperspirant product with the desired perspiration wetness and odor control.

The rheological solid personal care composition can comprise, or provide finished product that comprises, solubilized antiperspirant active at concentrations of from about 0.1 wt. % to about 35 wt. %, preferably from about 3 wt. % to about 20 wt. %, even more preferably from about 4 wt. % to about 19 wt. %, by weight of the composition. All such weight percentages are calculated on an anhydrous metal salt basis exclusive of water and any complexing or buffering agent such as glycine, glycine salts, or other complexing or buffering agent.

The solubilized antiperspirant active for use in the compositions of the present invention include any compound, composition or other material having antiperspirant activity. Preferred antiperspirant actives include astringent metallic salts, especially the inorganic and organic salts of aluminum, zirconium and zinc, as well as mixtures thereof. Particularly preferred are the aluminum and zirconium salts, such as aluminum halides, aluminum chlorohydrate, aluminum hydroxyhalides, zirconyl oxyhalides, zirconyl hydroxyhalides, and mixtures thereof.

Preferred aluminum salts for use in the antiperspirant compositions include those which conform to the formula:

Al₂(OH)ₐCl_{b}·x H₂O

wherein a is from about 2 to about 5; the sum of a and b is about 6; x is from about 1 to about 6; and wherein a, b, and x may have non-integer values. Particularly preferred are the aluminum chlorhydroxides referred to as “⅚ basic chlorhydroxide”, wherein a=5, and “⅔ basic chlorhydroxide”, wherein a=4.

Preferred zirconium salts for use in the antiperspirant compositions include those which conform to the formula:

ZrO(OH)₂₋ₐClₐ·x H₂O

wherein a is any number having a value of from about 0 to about 2; x is from about 1 to about 7; and wherein a and x may both have non-integer values. Particularly preferred zirconium salts are those complexes which additionally contain aluminum and glycine, commonly known as ZAG complexes. These ZAG complexes contain aluminum chlorhydroxide and zirconyl hydroxy chloride conforming to the above described formulas.

### Teeth Treatment Actives

The composition may comprise a water-soluble fluoride compound in an amount sufficient to give a fluoride ion concentration in the composition, and/or when it is used of from about 0.0025% to about 5.0% by weight, preferably from about 0.005% to about 2.0% by weight, to provide anticaries effectiveness. A wide variety of fluoride ion-yielding materials can be employed as sources of soluble fluoride in the present compositions. Examples of suitable fluoride ion-yielding materials are found in U.S. Pat. No. 3,535,421, Oct. 20, 1970 to Briner et al. and U.S. Pat. No. 3,678,154, July 18. 1972 to Widder et al. Representative fluoride ion sources include stannous fluoride, sodium fluoride, potassium fluoride, sodium monofluorophosphate, indium fluoride and many others. Stannous fluoride and sodium fluoride are preferred, as well as mixtures thereof.

### Topical Drug Actives

The rheological solid personal care composition may comprise topical drug actives which are insoluble. In some aspects, the rheological solid personal care composition can comprise from about 0.01 to about 20 wt. % of a topical drug active, alternatively from about 0.025 to about 10 wt. %, alternatively from about 0.1 to about 7 wt. %, alternatively from about 0.25 to about 5 wt. %, alternatively from about 1 to about 3 wt. %, all by weight of the rheological solid personal care composition. Non-limiting examples of topical drug actives can include analgesics like methyl salicylate, ibuprofen, and diclofenac sodium, melatonin, capsaicin, capsicum, camphor, menthol, anesthetics like benzocaine, corticosteroids like hydrocortisone and hydrocortisone acetate, and combinations thereof.

### Aqueous Phase

The rheological solid personal care composition contains a majority of water. However, other components can be optionally dissolved in the water to create an aqueous phase. These components are referred to as soluble active agents. Such soluble active agents can include, but are not limited to, catalysts, activators, peroxides, enzymes, antimicrobial agents, preservatives, salts such as sodium chloride, polyols, soluble pharmaceutical actives, and combinations thereof. The crystallizing agent and insoluble active agents are dispersed in the aqueous phase. The suspension agent may be dissolved in the aqueous phase (as with gums and other soluble polymers) or may be dispersed in the aqueous phase (as with clay particles).

### Catalysts

In some aspects, soluble active agents can include one or more metal catalysts. In some aspects, the metal catalyst can include one or more of dichloro-1.4-diethyl-1,4,8,11-tetraaazabicyclo[6.6.2] hexadecane manganese (II); and dichloro-1,4-dimethyl-1,4,8,11-tetraaazabicyclo[6.6.2]hexadecane manganese (II). In some aspects, the non-metal catalyst can include one or more of 2-[3-[(2-hexyldodecyl)oxy]-2-(sulfooxy)propyl]-3,4-dihydroisoquinolinium, inner salt; 3,4-dihydro-2-[3-[(2-pentylundecyl)oxy]-2-(sulfooxy)propyllisoquinolinium, inner salt; 2-[3-[(2-butyldecyl)oxy]-2-(sulfooxy)propyl]-3,4-dihydroisoquinolinium, inner salt; 3,4-dihydro-2-[3-(octadecyloxy)-2-(sulfooxy)propyllisoquinolinium, inner salt; 2-[3-(hexadecyloxy)-2-(sulfooxy)propyl]-3,4-dihydroisoquinolinium, inner salt; 3,4-dihydro-2-[2-(sulfooxy)-3-(tetradecyloxy)propyllisoquinolinium, inner salt; 2-[3-(dodecyloxy)-2-(sulfooxy)propyl]-3,4-dihydroisoquinolinium, inner salt; 2-[3-[(3-hexyldecyl)oxy]-2-(sulfooxy)propyl]-3,4-dihydroisoquinolinium, inner salt; 3,4-dihydro-2-[3-[(2-pentylnonyl)oxy]-2-(sulfooxy)propyl]isoquinolinium, inner salt; 3,4-dihydro-2-[3-[(2-propylheptyl)oxy]-2-(sulfooxy)propyl]isoquinolinium, inner salt; 2-[3-[(2-butyloctyl)oxy]-2-(sulfooxy)propyl]-3,4-dihydroisoquinolinium, inner salt; 2-[3-(decyloxy)-2-(sulfooxy)propyl]-3,4-dihydroisoquinolinium, inner salt; 3,4-dihydro-2-[3-(octyloxy)-2-(sulfooxy)propyl]isoquinolinium, inner salt; and 2-[3-[(2-ethylhexyl)oxy]-2-(sulfooxy)propyl]-3,4-dihydroisoquinolinium, inner salt.

### Activators

In some aspects, soluble active agents can include one or more activators. In some aspects, the activator can include one or more of tetraacetyl ethylene diamine (TAED); benzoylcaprolactam (BzCL); 4-nitrobenzoylcaprolactam; 3-chlorobenzoylcaprolactam; benzoyloxybenzenesulphonate (BOBS); nonanoyloxybenzene-sulphonate (NOBS); phenyl benzoate (PhBz); decanoyloxybenzenesulphonate (C₁₀-OBS); benzoylvalerolactam (BZVL); octanoyloxybenzenesulphonate (C₈-OBS); perhydrolyzable esters; 4-[N-(nonaoyl) amino hexanoyloxy]-benzene sulfonate sodium salt (NACA-OBS); dodecanoyloxybenzenesulphonate (LOBS or C₁₂-OBS); 10-undecenoyloxybenzenesulfonate (UDOBS or CH₁₁-OBS with unsaturation in the 10 position); decanoyloxybenzoic acid (DOBA); (6-oclanamidocaproyl)oxybenzenesulfonate; (6-nonanamidocaproyl) oxybenzenesulfonate; and (6-decanamidocaproyl)oxybenzenesulfonate.

### Peroxy-Carboxylic Acids

In some aspects, soluble active agent can include one or more preformed peroxy carboxylic acids. In some aspects, the peroxy carboxylic acids can include one or more of peroxymonosulfuric acids; perimidic acids; percabonic acids; percarboxilic acids and salts of said acids; phthalimidoperoxyhexanoic acid; amidoperoxyacids; 1,12-diperoxydodecanedioic acid; and monoperoxyphthalic acid (magnesium salt hexahydrate), wherein said amidoperoxyacids may include N,N′-terephthaloyl-di(6-aminocaproic acid), a monononylamide of either peroxysuccinic acid (NAPSA) or of peroxyadipic acid (NAPAA), or N-nonanoylaminoperoxycaproic acid (NAPCA).

In some aspects, water-based and/or water-soluble benefit agents can include one or more diacyl peroxide. In some aspects, the diacyl peroxide can include one or more of dinonanoyl peroxide, didecanoyl peroxide, diundecanoyl peroxide, dilauroyl peroxide, and dibenzoyl peroxide, di-(3,5,5-trimethyl hexanoyl) peroxide, wherein said diacyl peroxide can be clatharated.

### Peroxides

In some aspects, soluble active agents can include one or more hydrogen peroxide. In some aspects, hydrogen peroxide source can include one or more of a perborate, a percarbonate, a peroxyhydrate, a peroxide, a persulfate, and mixtures thereof, in one aspect said hydrogen peroxide source may comprise sodium perborate, in one aspect said sodium perforate may comprise a mono-or tetra-hydrate, sodium pyrophosphate peroxyhydrate, urea peroxyhydrate, trisodium phosphate peroxyhydrate, and sodium peroxide.

### Enzymes

In some aspects, soluble active agents can include one or more enzymes. In some aspects, the enzyme can include one or more of peroxidases, proteases, lipases, phospholipases, cellulases, cellobiohydrolases, cellobiose dehydrogenases, esterases, cutinases, pectinases, mannanases, pectate lyases, keratinases, reductases, oxidases, phenoloxidases, lipoxygenases, ligninases, pullulanases, tannases, pentosanases, glucanases, arabinosidases, hyaluronidase, chondroitinase, laccases, amylases, dnases, and combinations thereof.

### Sensate

In some aspects, soluble active agents can include one or more components that provide a sensory benefit. often called a sensate. Sensates can have sensory attributes such as a warming, tingling, or cooling sensation. Suitable sensates can include, for example, menthol, menthyl lactate, leaf alcohol, camphor, clove bud oil, eucalyptus oil, anethole, methyl salicylate, eucalyptol, cassia, 1-8 menthyl acetate, eugenol, oxanone, alpha-irisone, propenyl guaethol, thymol, linalool, benzaldehyde, cinnamaldehyde glycerol acetal known as “CGA”, N-[(ethoxycarbonyl)methyl)-p-menthane-3-carboxamide, known as “WS-5”, supplied by Renessenz-Symrise, and mixtures thereof.

In some aspects, the sensate comprises a coolant. The coolant can be any of a wide variety of materials. Included among such materials are carboxamides, menthol, ketals, diols, and mixtures thereof. Some examples of carboxamide coolants include, for example, paramenthan carboxyamide agents such as N-ethyl-p-menthan-3-carboxamide, known commercially as “WS-3”, N,2,3-trimethyl-2-isopropylbutanamide, known as “WS-23,” and N-(4-cyanomethylphenyl)-p-menthanecarboxamide, known as “G-180” and supplied by Givaudan. G-180 generally comes as a 7.5% solution in a flavor oil, such as spearmint oil or peppermint oil. Examples of menthol coolants include, for example, menthol; 3-1-menthoxypropane-1,2-diol, known as TK-10 and manufactured by Takasago; menthone glycerol acetal, known as “MGA” and manufactured by Haarmann and Reimer; and menthyl lactate, known as Frescolat® and manufactured by Haarmann and Reimer. The terms menthol and menthyl as used herein include dextro-and levorotatory isomers of these compounds and racemic mixtures thereof.

In some aspects, the sensate comprises a coolant selected from the group consisting of menthol: 3-1-menthoxypropane-1,2-diol; menthyl lactate; N,2,3-trimethyl-2-isopropylbutanamide; N-ethyl-p-menthan-3-carboxamide; N-(4-cyanomethylphenyl)-p-menthanecarboxamide; menthyl ethylamido oxalate; and combinations thereof. In some aspects, the sensate comprises menthol; N,2,3-trimethyl-2-isopropylbutanamide; N-(4-cyanomethylphenyl)-p-menthanecarboxamide; menthyl ethylamido oxalate, and combinations thereof.

In some aspects, the sensate comprises a warming sensates. Non-limiting examples of warming sensates can include vanillyl alcohol n-butyl ether (sold as TK-1000 by Takasago International), vanillyl butyl ether (commercially available as HotFlux® from Corum, Inc., Taipei, Taiwan), capsaicin, nonivamide, ginger, capsicum (commercially available as Vegetol® Capsicum LC481 from Gattefossé, Lyon, France), and combinations thereof.

In some aspects, the sensate comprises a tingling sensate. Non-limiting examples of tingling sensates can include sichuan pepper, hydroxy alpha sanshool, jambu extracts, spilanthol, and combinations thereof. A suitable sensory enhancer can include a neuro-soother such as Mariliance™ available from Givaudan, Vernier, Switzerland.

One advantage to including a sensate is that they can provide a topical sensory effect. When the rheological solid personal care composition having one or more sensates is applied to the skin, it can provide an on-skin sensation that can work in unison with the smell to provide an increased perception of product strength.

The rheological solid personal care composition can comprise from about 0.001 to about 1.5 wt. % of a sensate, alternatively from about 0.01 to about 1 wt. %, alternatively from about 0.1 to about 0.75 wt. %, alternatively from about 0.2 wt. % to about 0.5 wt. %, all by weight of the rheological solid personal care composition.

### Surfactant

In some aspects, soluble active agents can include one or more surfactants. These include cationic, anionic, and non-surfactants. This includes fabric conditioner softener surfactants and cleaning surfactants.

### Antimicrobial Compounds

In some aspects, soluble active agents can include an effective amount of a compound for reducing the number of viable microbes in the air or on inanimate surfaces. Antimicrobial compounds are effective on gram negative or gram positive bacteria or fungi typically found on indoor surfaces that have contacted human skin or pets such as couches, pillows, pet bedding, and carpets. Such microbial species include Klebsiella pneumoniae, Staphylococcus aureus, Aspergillus niger, Klebsiella pneumoniae, Steptococcus pyogenes, Salmonella choleraesuis, Escherichia coli, Trichophyton mentagrophytes, and Pseudomonas aeruginosa. The antimicrobial compounds may also be effective at reducing the number of viable viruses such H1-N1, Rhinovirus, Respiratory Syncytial, Poliovirus Type 1, Rotavirus, Influenza A, Herpes simplex types 1 & 2, Hepatitis A, and Human Coronavirus.

Antimicrobial compounds suitable in the rheological solid composition can be any organic material which will not cause damage to fabric appearance (e.g., discoloration, coloration such as yellowing, bleaching). Water-soluble antimicrobial compounds include organic sulfur compounds, halogenated compounds, cyclic organic nitrogen compounds, low molecular weight aldehydes, quaternary compounds, dehydroacetic acid, phenyl and phenoxy compounds, or mixtures thereof.

A quaternary compound may be used. Examples of commercially available quaternary compounds suitable for use in the rheological solid composition are Barquat® available from Lonza Corporation; and didecyl dimethyl ammonium chloride quat under the trade name Bardac® 2250 from Lonza Corporation.

The antimicrobial compound may be present in an amount from about 500 ppm to about 7000 ppm, alternatively about 1000 ppm to about 5000 ppm, alternatively about 1000 ppm to about 3000 ppm, alternatively about 1400 ppm to about 2500 ppm, by weight of the rheological solid personal care composition.

### Preservatives

In some aspects, soluble active agents can include a preservative. The preservative may be present in an amount sufficient to prevent spoilage or prevent growth of inadvertently added microorganisms for a specific period of time, but not sufficient enough to contribute to the odor neutralizing performance of the rheological solid composition. In other words, the preservative is not being used as the antimicrobial compound to kill microorganisms on the surface onto which the rheological solid composition is deposited in order to eliminate odors produced by microorganisms. Instead, it is being used to prevent spoilage of the rheological solid personal care composition in order to increase the shelf-life of the rheological solid personal care composition.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis{N′-[1-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hüls America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation; 1,2-Benzisothiazolin-3-one; Acticide MBS.

Suitable levels of preservative are from about 0.0001 wt. % to about 0.5 wt. %, alternatively from about 0.0002 wt. % to about 0.2 wt. %, alternatively from about 0.0003 wt. % to about 0.1 wt. %, by weight of the rheological solid personal care composition.

The rheological solid personal care composition may include an aqueous carrier. The aqueous carrier which is used may be distilled, deionized, or tap water. Water may be present in any amount for the rheological solid personal care composition to be an aqueous solution. Water may be present in an amount of about 85 wt. % to 99.5 wt. %, alternatively about 90 wt. % to about 99.5 wt. %, alternatively about 92 wt. % to about 99.5 wt. %, alternatively about 95 wt. %, by weight of the rheological solid personal care composition. Alternatively, water may be present in an amount of about 55 wt. % to about 99.5 wt. %, alternatively from about 60 wt. % to about 99.5%, alternatively from about 65 wt. % to about 95%, alternatively from about 70 wt. % to about 95 wt. %, alternatively from about 75 wt. % to about 90 wt. %, all by weight of the rheological solid personal care composition.

Water containing a small amount of low molecular weight monohydric alcohols, e.g., ethanol, methanol, and isopropanol, or polyols, such as ethylene glycol and propylene glycol, can also be useful. However, the volatile low molecular weight monohydric alcohols such as ethanol and/or isopropanol should be limited since these volatile organic compounds will contribute both to flammability problems and environmental pollution problems. If small amounts of low molecular weight monohydric alcohols are present in the rheological solid composition due to the addition of these alcohols to such things as perfumes and as stabilizers for some preservatives, the level of monohydric alcohol may about 1 wt. % to about 5 wt. %, alternatively less than about 6 wt. %, alternatively less than about 3 wt. %, alternatively less than about 1 wt. %, by weight of the rheological solid personal care composition.

### Adjuvants

Adjuvants can be added to the rheological solid personal care composition herein for their known purposes. Such adjuvants include, but are not limited to, water soluble metallic salts, including zinc salts, copper salts, and mixtures thereof; antistatic agents; insect and moth repelling agents; colorants; antioxidants; aromatherapy agents, and mixtures thereof.

The compositions of the present invention can also comprise any additive usually used in the field under consideration. For example, non-encapsulated pigments, film forming agents, dispersants, antioxidants, essential oils, preserving agents, fragrances, liposoluble polymers that are dispersible in the medium, fillers, neutralizing agents, silicone elastomers, cosmetic and dermatological oil-soluble active agents such as, for example, emollients, moisturizers, vitamins, anti-wrinkle agents, essential fatty acids, sunscreens, and mixtures thereof can be added.

### Solvents

The rheological solid personal care composition can contain a solvent. Non-limiting examples of solvents can include ethanol, glycerol, propylene glycol, polyethylene glycol 400, polyethylene glycol 200, and mixtures thereof. In some aspects, the rheological solid personal care composition can comprise from about 0.5 wt. % to about 15 wt. % solvent, alternatively from about 1.0 wt. % to about 10 wt. % solvent, alternatively from about 1.0 wt. % to about 8.0 wt. % solvent, alternatively from about 1 wt. % solvent to about 5 wt. % solvent, all by weight of the rheological solid personal care composition.

### Vitamins

As used herein, “xanthine compound” means one or more xanthines, derivatives thereof, and mixtures thereof. Xanthine compounds that can be useful herein include, but are not limited to, caffeine, xanthine, 1-methyl xanthine, theophylline, theobromine, derivatives thereof, and mixtures thereof. Among these compounds, caffeine is preferred in view of its solubility in the composition. The composition can contain from about 0.05 wt. %, preferably from about 2.0 wt. %, more preferably from about 0.1 wt. %, still more preferably from about 1.0 wt. %, and to about 0.2 wt. %, preferably to about 1.0 wt. %, more preferably to about 0.3 wt. % by weight of a xanthine compound.

As used herein, “vitamin B3 compound” means a one or more compounds having the formula:

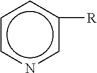

wherein R is —CONH₂ (i.e., niacinamide), —COOH (i.e., nicotinic acid) or —CH₂OH (i.e., nicotinyl alcohol); derivatives thereof; mixtures thereof; and salts of any of the foregoing.

Exemplary derivatives of the foregoing vitamin B3 compounds include nicotinic acid esters, including non-vasodilating esters of nicotinic acid (e.g, tocopherol nicotinate, and myristyl nicotinate), nicotinyl amino acids, nicotinyl alcohol esters of carboxylic acids, nicotinic acid N-oxide and niacinamide N-oxide. The composition can contain from about 0.05 wt. %, preferably from about 2.0 wt. %, more preferably from about 0.1 wt. %, still more preferably from about 1.0 wt. %, and to about 0.1 wt. %, preferably to about 0.5 wt. %, more preferably to about 0.3 wt. %, by weight of a vitamin B3 compound.

As used herein, the term “panthenol compound” is broad enough to include panthenol, one or more pantothenic acid derivatives, and mixtures thereof. Panthenol and its derivatives can include D-panthenol ([R]-2,4-dihydroxy-N-[3-hydroxypropyl)]-3,3-dimethylbutamide), DL-panthenol, pantothenic acids and their salts, preferably the calcium salt, panthenyl triacetate, royal jelly, panthetine, pantotheine, panthenyl ethyl ether, pangamic acid, pantoyl lactose, vitamin B complex. or mixtures thereof. The composition can contain from about 0.01 wt. %, preferably from about 0.02 wt. %, more preferably from about 0.05 wt. %, and to about 3 wt. %, preferably to about 1 wt. %, more preferably to about 0.5 wt. % by, weight of a panthenol compound.

### Salts

In some aspects, the rheological solid personal care composition may comprise a salt, which can help with thermal stability. Non-limiting examples of salts can include sodium chloride, sodium sulfate, and combinations thereof. In some aspects, the rheological solid personal care composition can comprise from about 0.1 to about 10 wt. % of a salt, alternatively from about 1 to about 7 wt. %, alternatively 3 to about 5 wt. %, all by weight of the rheological solid personal care composition.

### Soluble Pharmaceutical Actives

The rheological solid personal care composition can comprise a soluble pharmaceutical active. In some aspects, the rheological solid personal care composition can comprise from about 0.1 to about 5 wt. % of a soluble pharmaceutical active, alternatively from about 0.25 to about 3 wt. %, alternatively 0.5 to about 1.5 wt. %, all by weight of the rheological solid personal care composition. Non-limiting examples of soluble pharmaceutical actives can include antihistamines, such as diphenhydramine hydrochloride and tripelennamine hydrochloride, anesthetics, such as lidocaine hydrochloride, dibucaine, pramoxine, and tetracaine, and combinations thereof.

### Consumer Product/Rheological Solid Personal Care Composition

In one aspect, the rheological solid personal care composition can provide at least temporarily cough suppression due to minor throat and bronchial irritation such as associated with the common cold. In one aspect, the rheological solid personal care composition can provide at least temporarily relief of minor aches and/or pains of muscles and/or joints. In one aspect, the rheological solid personal care composition can provide relief of nasal congestion.

The rheological solid personal care composition can be applied to the skin of a user on the back, throat, forehead, and/or chest. The user can place a desired amount of the rheological solid personal care composition on his or her skin and rub it in for about 5 seconds to about 3 minutes, alternatively for about 20 seconds to about 90 seconds, alternatively for about 30 seconds to about 60 seconds. In one example, the rheological solid personal care composition can be covered with a warm, dry cloth after application to the skin.

A dose of the rheological solid personal care composition can be applied to the skin and/or clothing once daily, or twice daily, or three times per day. In one aspect, a dose of the rheological solid personal care composition can be applied to the skin up to three times per day. The rheological solid personal care composition can be applied to the skin and/or clothing on a daily basis or only as needed. Preferably the rheological solid personal care composition is applied to and allowed to dry before subjecting to contact such as with clothing or other objects. The rheological solid personal care composition is preferably applied to the desired area that is dry or has been dried prior to application.

A dose of the rheological solid personal care composition can comprise from about 0.5 g to about 10 g, alternatively from about 1 g to about 8 g, alternatively from about 1.5 g to about 6 g, alternatively from about 3 g to about 4.5 g, alternatively about 7.5 g.

Another aspect of the present invention includes a method of providing one or more health benefits, cosmetic benefits, and/or consumer benefits by administering the rheological solid personal care composition to a user in need thereof. Non-limiting examples of the one or more health benefits can include providing relief of nasal congestion, suppressing a cough, providing relief of muscle aches and pain, improving the quality of sleep to a user suffering from a cold or flu, providing topical analgesic effects, providing relief from rash, pain, and/or dermatitis, and combinations thereof. Non-limiting examples of the one or more cosmetic benefits can include moisturizing, cleansing, beautifying, and combinations thereof. Non-limiting examples of the one or more consumer benefits can include providing soothing vapors, providing aromatherapy, promoting sleep, providing stress relief, energizing, providing calming and/or relaxing scents, and combinations thereof.

The compositions of the present invention make it possible to obtain superior consumer aesthetics without compromising stability. The preferred ratios and weight percentages identified above provide sufficient medium coverage of product without being perceived as dry or flakey and provide a nice smoothing/evening effect of the skin. They also provide a pleasant fresh feel on the skin upon application of the composition.

The present invention also envisages kits and/or prepackaged materials suitable for consumer use containing one or more compositions according to the description herein. The packaging and application device for any subject of the invention may be chosen and manufactured by persons skilled in the art on the basis of their general knowledge; and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

The rheological solid personal care compositions of the present invention may also be combined with a device, such as a container, non-woven sheet or roller, given the soft-solid nature of the material. Such composition/device combinations can be used as consumer products for such diverse applications as skin cooling or vapor applicators (e.g. sticks, balls), non-woven webs (e.g. surface wipes, mops, toilet sheets), and fabric enhancers (e.g. fabric dryer sheets, fabric stain removal, fabric wrinkle reduction, fabric softeners).

### Properties

### Phase Stability

Phase stability, as used herein, is a measure of the effectiveness of the suspension agent(s) to prevent the sedimentation or creaming of dispersed active particles. A hot mixture of solubilized crystallizing agent in water at processing temperatures has a viscosity on the order of several milli-pascal seconds. At this stage, actives are added and dispersed as particles in the mixture. The active particles tend to cream (i.e. rise) or sediment (i.e. settle) in the time before crystallization of the crystallizing agent, leading to consumer-unacceptable separation of the materials. The suspension agent(s) prevent bulk separation of dispersed active particles during crystallization and allows a mesh of fiber-like crystalline particles to entrain the dispersed active particles. Not wishing to be bound by theory, it is believed that the suspension agent(s) either increases the suspension viscosity or enables a yield stress to the mixture that prevents active particle separation. A phase stability value of ‘0’ is not preferred, a value of ‘1’ is preferred values, and a value of ‘2’ is most preferred. Phase stability is determined using the PHASE STABILITY TEST METHOD, as described below.

### Stability Temperature

Stability temperature, as used herein, is the temperature at which most or all of the crystallizing agent completely dissolves into an aqueous phase, such that a composition no longer exhibits a stable solid structure and may also be considered a liquid. In some aspects, the minimal stability temperature may be from about 30° C. to about 95° C., about 40° C. to about 90° C., about 50° C. to about 80° C., or from about 60° C. to about 70° C., as these temperatures are typical in a supply chain. Stability temperature can be determined using the THERMAL STABILITY TEST METHOD, as described below.

### Firmness

Depending on the intended application, such as a stick, firmness of the composition may also be considered. The firmness of a composition may, for example, be expressed in Newtons of force. For example, compositions of the present invention comprising 1-3 wt % crystallizing agent may give values of about 4—12 N, in the form of a solid stick or coating on a sheet. As is evident, the firmness of the composition according to embodiments of the present invention may, for example, be such that the composition is advantageously self-supporting and can release liquids and/or actives easily to form a satisfactory deposit on a surface, such as the skin and/or superficial body growths, such as keratinous fibers. In addition, this firmness may impart good impact strength to the inventive compositions, which may be molded or cast, for example, in stick or sheet form, such as a wipe or dryer sheet product. The rheological solid personal care composition may also be transparent or clear, including for example, a composition without pigments. Preferred firmness is between about 0.1 N and about 50.0 N, more preferably between about 0.5 N—about 40.0 N, more preferably between about 1.0 N—about 30.0 N, and most preferably between about 2.5 N—about 15.0 N. The firmness may be measured using the FIRMNESS TEST METHOD, as described below.

### Liquid Expression

Depending on the intended application, such as a stick, liquid expression of the composition may also be considered. This is a measure of the amount of work need per unit volume to express water from the compositions, with larger values meaning it becomes more difficult to express water. A low value might be preferred, for example, when applying the composition to the skin. A high value might be preferred, for example, when applied to a substrate that requires ‘dry-to-the-touch-but-wet-to-the-wipe’ properties. Preferred values are between about 100 J m-3 and about 6000 J m-3, alternatively between about 100 J m-3 and about 3000 J m-3, alternatively between about 300

J m-3 and about 2000 J m-3, alternatively between about 500 J m-3 and about 1500 J m-3. The liquid expression may be measured using the WATER-EXPRESSION TEST METHOD, as described herein.

### Firmness Test Method

All samples and procedures are maintained at room temperature (25±3° C.) prior to and during testing, with care to ensure little or no water loss.

All measurements were made with a TA-XT2 Texture Analyzer (Texture Technology Corporation, Scarsdale, N.Y., U.S.A.) outfitted with a standard 45° angle penetration cone tool (Texture Technology Corp., as part number TA-15).

To operate the TA-XT2 Texture Analyzer, the tool is attached to the probe carrier arm and cleaned with a low-lint wipe. The sample is positioned and held firmly such that the tool will contact a representative region of the sample. The tool is reset to be about 1 cm above the product sample.

The sample is re-position so that the tool will contact a second representative region of the sample. A run is done by moving the tool at a rate of 2 mm/second exactly 10 mm into the sample. The “RUN” button on the Texture Analyzer can be pressed to perform the measurement. A second run is done with the same procedure at another representative region of the sample at sufficient distance from previous measurements that they do not affect the second run. A third run is done with the same procedure at another representative region of the sample at sufficient distance from previous measurements that they do not affect the third run.

The following Firmness values are returned from this measurement:

If the mixture fails to crystallize completely (e.g. remains clear or mushy) at Room Temperature, return a value of “NOT SOLID”; if the mixture is in excess of 48 N and too hard to measure, return a value of “TOO HARD”; otherwise a numeric value which is the average of the maximum value of three measurements is returned.

### Thermal Stability Test Method All samples and procedures are maintained at room temperature (25±3° C.) prior to testing.

Sampling is done at a representative region on the sample, in two steps. First, a spatula is cleaned with a laboratory wipe and a small amount of the sample is removed and discarded from the top of the sample at the region, to create a small square hole about 5 mm deep. Second, the spatula is cleaned again with a clean laboratory wipe, and a small amount of sample is collected from the square hole and loaded into DSC pan.

The sample is loaded into a DSC pan. All measurements are done in a high-volume-stainless-steel pan set (TA part #900825.902). The pan, lid and gasket are weighed and tared on a Mettler Toledo MT5 analytical microbalance (or equivalent). The sample is loaded into the pan with a target weight of 20 mg (+/−10 mg) in accordance with manufacturer's specifications, taking care to ensure that the sample is in contact with the bottom of the pan. The pan is then sealed with a TA High Volume Die Set (TA part #901608.905). The final assembly is measured to obtain the sample weight.

The sample is loaded into TA Q Series DSC in accordance with the manufacture instructions. The DSC procedure uses the following settings: 1) equilibrate at 25° C.; 2) mark end of cycle 1; 3) ramp 1.00° C./min to 90.00° C.; 4) mark end of cycle 3; then 5) end of method; Hit run.

The Stability Temperature is determined as the maximum peak value of the highest temperature peak. If Stability Temperature cannot be measured because the sample is liquid or the thermal stability is too low/too high to measure, then a sample is assigned a value of ‘NM’.

### Water-Expression Test Method

All samples and procedures are maintained at room temperature (25±3° C.) prior to testing. Measurements for the determination of Water-Expression were made with a TA Discovery HR-2 Hybrid Rheometer (TA Instruments, New Castle, Delaware, U.S.A.) and accompanying TRIOS software version 3.2.0.3877, or equivalent. The instrument is outfitted with a DHR Immobilization Cell (TA Instrument) and 50 mm flat steel plate (TA Instruments). The calibration is done in accordance with manufacturer's recommendations, with special attention to measuring the bottom of the DHR Immobilization Cell, to ensure this is established as gap=0.

Samples are prepared in accordance with EXAMPLE procedures. It is critical that the sample be prepared in Speed Mixer containers (Flak-Tech, Max 60 Cup Translucent, Cat #501 222t), so that the diameter of the sample matches the diameter of the HR-2 Immobilization Cell. The sample is released from the containers by running a thin spatula between the edge of the container and the sample. The container is gently turned over and placed on a flat surface. A gentle force is applied to the center of the bottom of the overturned container, until the sample releases and gently glides out of the container. The sample is carefully placed in the center ring of the DHR Immobilization Cell. Care is used to ensure that the sample is not deformed and re-shaped through this entire process. The diameter of the sample should be slightly smaller than the inner diameter of the ring. This ensures that force applied to the sample in latter steps does not significantly deform the cylindrical shape of the sample, instead allowing the fluid to escape through the bottom of the sample. This also ensures that any change in the height of the sample for the experiment is equivalent to the amount of aqueous phase expressed during the test. At the end of the measurement, one should confirm that the aqueous phase is indeed expressed from the sample through the measurement, by looking for water in the effluent tube connected to the Immobilization Cell. If no aqueous phase is observed, the sample is deemed not to express water and is not inventive.

Set the instrument settings as follows. Select Axial Test Geometry. Then, set “Geometry” options: Diameter=50 mm; Gap=45000 um; Loading Gap=45000 um; Trim Gap Offset=50 um; Material=‘Steel’; Environmental System=“Peltier Plate”. Set “Procedure” options: Temperature=25° C.; Soak Time=0 sec; Duration=2000 sec; Motor Direction=“Compression”; Constant Linear Rate=2 um sec-1; Maximum Gap Change=0 um; Torque=0 uN·m; Data Acquisition=‘save image’ every 5 sec.

Manually move the steel tool within about 1000 um of the surface of the sample, taking care that the tool does not touch the surface. In the “Geometry” options, reset Gap to this distance.

Start the run.

The data is expressed in two plots:
1) Plot 1: Axial Force (N) on the left-y-axis and Step Time(s) on the x-axis;
2) Plot 2: Gap (um) on the right-y-axis and Step Time(s) on the x-axis.

The Contact Time—T(contact), is obtained from Plot 1. The T(contact) is defined as the time when the tool touches the top of the sample. The T(contact) is the Step Time when the first Axial

Force data point exceeds 0.05 N.

The Sample Thickness—L, is the gap distance at the Contact Time, and expressed in units of meters.

The Time of Compression—T(compression), is the Step Time at which the gap is 0.85*L, or 15% of the sample.

The Work required to squeeze the water from the structure is the area under the Axial Force curve in Plot 1 between T(contact) and T(compression) multiplied by Constant Linear Rate, or 2e-6 m s-1 normalized by dividing the total volume of expressed fluids, and is expressed in units of Joules per cubic meter (J m-3).

If Water-Expression cannot be measured because the sample is a rheological solid but too soft to handle for testing, then a sample is assigned a value of ‘SOFT’.

### Phase Stability Test Method

Samples are prepared in accordance with EXAMPLE procedures.

For the examples that contain beads (Examples 1-6), the samples are separated into two fractions each placed into a container (Flak-Tech, Max 60 Cup Translucent, Cat #501 222t). Both containers are placed in an oven (Yamato, DKN 400; Yamato Scientific Co., Ltd., Tokyo, Japan, or equivalent) set to 60° C. for one hour. The containers are then placed on a bench top at room temperature (25° C.±3° C.). ‘Separation’ in the samples describes the creaming and/or sedimentation of the Microspheres.

Each of the samples is visually inspected for phase stability and graded based on the follow:
(most preferred) A grade of “2” is given if the composition appeared stable with no discernable separation of the beads (i.e. uniform);
(preferred) A grade of “1” is given if the preparation appeared with no more than 25% by number of the tracer beads on the top or bottom of the composition;
(not preferred) A grade of “0” is given if the composition appeared unstable as evident by nearly complete separation of the beads with more than 75% by number on the top and bottom of the composition.

For the examples that not contain beads (Examples 7-10), the entire sample is placed into a container (Flak-Tech, Max 60 Cup Translucent, Cat #501 222t) and placed in an oven (Yamato, DKN 400; Yamato Scientific Co., Ltd., Tokyo, Japan, or equivalent) set to 60° C. for one hour. The containers are placed on a bench top at room temperature (25±3° C.). ‘Separation’ in the samples describes the creaming and/or sedimentation of the insoluble active particles.

Each of the samples is visually inspected for phase stability and graded based on the follow:
(most preferred) A grade of “2” is given if the composition appeared stable with no discernable or visual separation of the insoluble active particles;
(preferred) A grade of “1” is given if the preparation appeared with only a few drops (estimated less than 25 wt % of the total amount of added insoluble active agent) on the top and/or bottom of the composition. In some compositions, this may result in a ‘slick’ appearance on the surface;
(not preferred) A grade of “0” is given if the compositions appeared unstable as evident by nearly complete separation of the insoluble active agent on the top or the bottom of the composition (estimated less than 75 wt % of the total amount of added insoluble active agent). In the case of oils, the amounts are sufficient to have the oil visually flow when the sample is turned sideways.

### EXAMPLES

### MATERIALS LIST

(1) Euxyl PE 9010 (EP)-Schülke & Mayr GmbH, Norderstedt, Germany, PE 9010 preservative lot 1501226
(2) SymDiol 68 (S68)—Symrise, Holzminden, Germany, Symdiol® 68 preservative lot 10300094
(3) Water—Millipore, Burlington, MA (18 m-ohm resistance)
(4) Sodium Myristate NaM—TCI Chemicals, Cambridge, MA, Cat. #M0483
(5) Xanthan Gum (x-gum)—CPK, Denmark, Keltrol 1000, LOT 6J3749K
(6) Konjac Gum (k-gum)—FMC Corporation, Philadelphia, PA, Nutricol® XP 3464, FMC, LOT 1192605
(7) Probe Particle Microspheres—Cospheric LLC, Santa Barbra, CA, UVPMS-BG-1.00 500-600 um
(8) Sodium Palmitate (NaP)—TCI Chemicals, Cambridge, MA, Cat. #P0007
(9) Sodium Stearate (NaS)—TCI Chemicals, Cambridge, MA, Cat. #S0081
(10) Starch-Spectrum, New Brunswick, NJ, Cat #9005-25-8
(11) Peppermint Oil—MFR Ungerer, Bethlehem, PA, lot no: 10059257SP-006
(12) Coconut Oil—Nature's Oil, Streetsboro, Ohio, Bulk Apothecary, SKU: bna-513
(13) PMC—Encapsys, Wisconsin, USA, Heavenly Powder PA PMC Slurry, lot no: 201810456
(14) L-Menthol
(15) Nutmeg Oil
(16) Camphor
(17) Eucalyptus Oil
(18) Cedar Leaf Oil FCC
(19) Turpentine Containing Antioxidant
(20) Thymol NF
(21) Sodium Chloride (NaCl)—VWR, Cat #BDH9286-500G
(22) Petrolatum—Calumet Specialty Products, Indianapolis, IN, Cat. #PEN1722-00-C
(23) Glycerol—Alfa Aesar, Cat #A16205
(24) Rheocrysta c-2sp—Iwase Csofa USA Inc., Fort Lee, NJ, Cat. #7UA/56203
(25) Laponite Suspension—Laponite XIG, BYK Additives & Instruments, Louisville, KY, Cat. # 13-235

### Stock Solutions

### (A1) Preparation of 1 wt. % Xanthan Gum Stock (X-gum Stock)

0.202 grams Euxyl PE 9010 (1), 0.305 grams SymDiol 68 (2) and 49.007 grams of water (3) were added to a Max 60 Speed Mixer cup (Flak-Tech, Max 60 Cup Translucent, Cat #501 222t). 0.502 grams xanthan gum (5) were added to the cup. The cup was placed in the Speed Mixer (Flak-Tech) at 2700 rpm for 150 seconds. Samples were allowed to sit for 2 hours and then Speed Mixed a second time for 2700 rpm for 150 seconds.

### (A2) Preparation of 1 wt. % Konjac Gum Stock (K-gum Stock)

0.201 grams Euxyl PE 9010 (1), 0.301 grams SymDiol 68 (2) and 49.001 grams of water (3) were added to a Max 60 Speed Mixer cup (Flak-Tech, Max 60 Cup Translucent, Cat #501 222t). 0.503 grams konjac gum (6) were added to the cup. The cup was placed in the Speed Mixer at 2700 rpm for 150 seconds. Samples were allowed to sit for 2 hours and then Speed Mixed a second time for 2700 rpm for 150 seconds.

### EXAMPLES

### Example 1

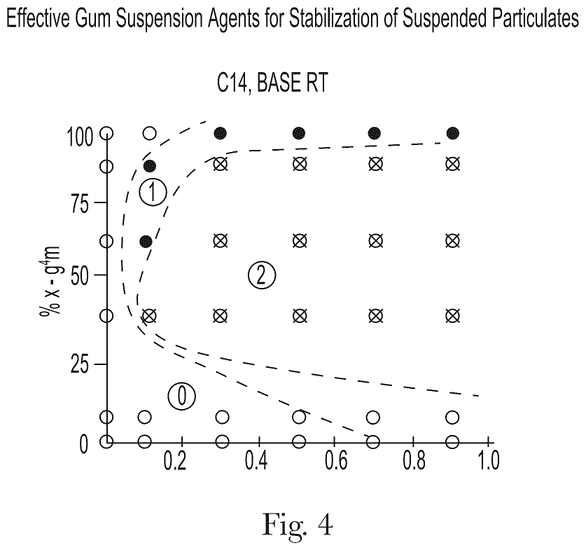

Samples A-AE use suspension agents made of a blend of gums for the stabilization of suspended insoluble active particles (FIG. 4). In these compositions, the suspension agent was composed of differing amounts of x-gum and k-gum, at 5 wt. % of the crystallizing agent, sodium myristate. FIG. 4 plots the total weight of the gum (i.e. weight x-gum+weight k-gum) along the x-axis and plots the weight percentage of the x-gum (i.e. weight x-gum/(weight x-gum+weight k-gum)) along the y-axis where each point in the plot represents a phase stability outcome of the compositions in Tables 1-8 below. ‘X’ markers indicate compositions that have a stability grade of ‘0’ as determined by the PHASE STABILITY TEST METHOD, and are comparative compositions; ‘□’ markers indicate compositions that have a stability grade of ‘1’ as determined by the PHASE STABILITY TEST METHOD, and are preferred inventive compositions; ‘O’ markers indicate compositions that have a stability grade of ‘2’ as determined by the PHASE STABILITY TEST METHOD, and are most preferred compositions. The data show that certain compositions of suspension agents are more preferred for stabilizing insoluble actives. Exclusion of suspension agent from the composition always resulted in stability grades of ‘0’. Not wishing to be bound by theory, this is due the presence of yield stress in the preparation created by suspension agents during the cooling process. Surprisingly, many of the compositional limits vary substantially owing to the presence of the crystallizing agent. Tables 1-8 also contain firmness (FIRMNESS TEST METHOD), temperature (THERMAL STABILITY TEST METHOD) and work (WATER-EXPRESSION TEST METHOD) data for representative comparative and inventive compositions. These data demonstrate that the prototypes exhibit the desired properties for these rheological solid personal care compositions, even in the presence of the suspension agents.

### Preparation of Compositions

Compositions were prepared using a heated mixing device. An overhead mixer (IKA Works Inc, Wilmington, NC, model RW20 DMZ) and a three-blade impeller design was assembled. All preparations were heated on a heating-pad assembly (VWR, Radnor, PA, 7×7 CER Hotplate, cat. no. NO97042-690) where heating was controlled with an accompanying probe. All preparations were done in a 250 ml stainless steel beaker (Thermo Fischer Scientific, Waltham, MA).

The NaM/water solution was prepared by first adding the preservatives (1, 2). Water (3), and Na-Myristate (4) were then added to the beaker. The beaker was placed on the heating-pad assembly. The overhead stirrer was placed in the beaker and set to rotate at 100 rpm. The heater was set at 80° C. The preparation was heated to 80° C. The heat was turned off and the preparation allowed to cool to 60° C.

The final composition was prepared by adding 1% Xanthan Gum Stock (A1) to the Na-M/Water solution, and the stirring rate increased to 300-350 rpm. Once the xanthan gum was completely added and mixed, the 1% Konjac Gum Stock (A2) was added to the Na-M/Water/Xanthan solution, and the stirring rate was increased to 500-550 rpm. Then the solid benefit agents were added to the beaker with continuous stirring and allowed to completely disperse. The composition was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples. Representative data demonstrate that the prototypes exhibit the desired properties for these rheological solid compositions, even in the presence of the suspension agents.

TABLE 1

| | Sample A | Sample B | Sample C | | Sample D | |
|---|---|---|---|---|---|---|
| | Comparative | Comparative | Comparative | | Inventive | |
| (1) Euxyl PE | 0.404 g | 0.400 g | 0.403 | g | 0.400 | g |
| (2) S68 | 0.603 g | 0.600 g | 0.602 | g | 0.601 | g |
| (3) Water | 92.702 g | 90.702 g | 88.702 | g | 86.701 | g |
| (4) NaM | 5.002 g | 5.001 g | 5.003 | g | 5.002 | g |
| NaM wt % | 5.0% | 5.0% | 5.0% | | 5.0% | |
| (A1) X-gum Stock | — | — | — | | — | |
| (A2) K-gum Stock | 1.002 g | 3.005 g | 5.002 | g | 7.002 | g |
| Gum wt % | 0.01% | 0.03% | 0.05% | | 0.07% | |
| % X-gum | 0% | 0% | 0% | | 0% | |
| (7) Microspheres | 0.300 g | 0.302 g | 0.304 | g | 0.300 | g |
| Stability | 0 | 0 | 0 | | 1 | |
| Firmness | 7.90N | 9.78N | 9.80N | | 10.12N | |
| Temperature | — | — | 36.4° | C. | 36.7° | C. |
| Work | — | — | 894 | J m−3 | 1261 | J m−3 |

TABLE 2

| | Sample E | Sample F | | Sample G | Sample H |
|---|---|---|---|---|---|
| | Inventive | Comparative | | Comparative | Comparative |
| (1) Euxyl PE | 0.404 g | 0.404 | g | 0.404 g | 0.401 g |
| (2) S68 | 0.601 g | 0.602 | g | 0.603 g | 0.601 g |
| (3) Water | 84.703 g | 92.700 | g | 90.702 g | 88.701 g |
| (4) NaM | 5.000 g | 5.000 | g | 5.000 g | 5.000 g |
| NaM wt % | 5.0% | 5.0% | | 5.0% | 5.0% |
| (A1) X-gum Stock | — | 0.103 | g | 0.302 g | 0.502 g |
| (A2) K-gum Stock | 9.003 g | 0.900 | g | 2.702 g | 4.503 g |
| Gum wt % | 0.09% | 0.01% | | 0.03% | 0.05% |
| % X-gum | 0% | 10% | | 10% | 10% |
| (7) Microspheres | 0.300 g | 0.303 | g | 0.302 g | 0.304 g |
| Stability | 1 | 0 | | 0 | 0 |
| Firmness | — | 10.11N | | 10.00N | 8.52N |
| Temperature | — | 39.4° | C. | — | — |
| Work | — | 618 | J m−3 | — | — |

TABLE 3

| | Sample I | Sample J | Sample K | Sample L | |
|---|---|---|---|---|---|
| | Inventive | Inventive | Inventive | Inventive | |
| (1) Euxyl PE | 0.401 g | 0.400 g | 0.403 g | 0.401 | g |
| (2) S68 | 0.602 g | 0.602 g | 0.600 g | 0.603 | g |
| (3) Water | 86.700 g | 84.702 g | 92.701 g | 90.703 | g |
| (4) NaM | 5.001 g | 5.000 g | 5.001 g | 5.001 | g |
| NaM wt % | 5.0% | 5.0% | 5.0% | 5.0% | |
| (A1) X-gum Stock | 0.704 g | 0.901 g | 0.400 g | 1.204 | g |
| (A2) K-gum Stock | 6.303 g | 8.103 g | 0.602 g | 1.803 | g |
| Gum wt % | 0.07% | 0.09% | 0.01% | 0.03% | |
| % X-gum | 10% | 10% | 40% | 40% | |
| (7) Microspheres | 0.301 g | 0.300 g | 0.302 g | 0.302 | g |
| Stability | 1 | 1 | 2 | 2 | |
| Firmness | 10.19N | 9.67N | 11.54N | 11.24N | |
| Temperature | — | — | — | 41.5° | C. |
| Work | — | — | — | 1170 | J m−3 |

TABLE 4

| | Sample M | | Sample N | Sample O | | Sample P | |
|---|---|---|---|---|---|---|---|
| | Inventive | | Inventive | Inventive | | Inventive | |
| (1) Euxyl PE | 0.402 | g | 0.402 g | 0.403 | g | 0.402 | g |
| (2) S68 | 0.600 | g | 0.602 g | 0.604 | g | 0.600 | g |
| (3) Water | 88.703 | g | 87.703 g | 84.700 | g | 92.701 | g |
| (4) NaM | 5.001 | g | 5.003 g | 5.001 | g | 5.001 | g |
| NaM wt % | 5.0% | | 4.9% | 5.0% | | 5.0% | |
| (A1) X-gum Stock | 2.004 | g | 2.801 g | 3.601 | g | 0.654 | g |
| (A2) K-gum Stock | 3.003 | g | 4.704 g | 5.402 | g | 0.453 | g |
| Gum wt % | 0.05% | | 0.07% | 0.09% | | 0.01% | |
| % X-gum | 40% | | 40% | 40% | | 65% | |
| (7) Microspheres | 0.304 | g | 0.303 g | 0.302 | g | 0.302 | g |
| Stability | 2 | | 2 | 2 | | 1 | |
| Firmness | 10.86N | | 10.10N | 9.29N | | 10.02N | |
| Temperature | 40.3° | C. | — | 40.9° | C. | 38.9° | C. |
| Work | 1,934 | J m−3 | — | 1,523 | J m−3 | 1,719 | J m−3 |

TABLE 5

| | Sample Q | Sample R | | Sample S | Sample T |
|---|---|---|---|---|---|
| | Inventive | Inventive | | Inventive | Inventive |
| (1) Euxyl PE | 0.401 g | 0.403 | g | 0.403 g | 0.402 g |
| (2) S68 | 0.601 g | 0.602 | g | 0.604 g | 0.601 g |
| (3) Water | 90.700 g | 88.700 | g | 86.704 g | 84.700 g |
| (4) NaM | 5.000 g | 5.000 | g | 5.002 g | 5.000 g |
| NaM wt % | 5.0% | 5.0% | | 5.0% | 5.0% |
| (A1) X-gum Stock | 1.951 g | 3.251 | g | 4.551 g | 5.853 g |
| (A2) K-gum Stock | 1.053 g | 1.752 | g | 2.453 g | 3.151 g |
| Gum wt % | 0.03% | 0.05% | | 0.07% | 0.09% |
| % X-gum | 65% | 65% | | 65% | 65% |
| (7) Microspheres | 0.303 g | 0.303 | g | 0.301 g | 0.300 g |
| Stability | 2 | 2 | | 2 | 2 |
| Firmness | 9.53N | 9.02N | | 8.51N | 8.14N |
| Temperature | — | 39.0° | C. | — | — |
| Work | — | 2,073 | J m−3 | — | — |

TABLE 6

| | Sample U | Sample V | | Sample W | Sample X |
|---|---|---|---|---|---|
| | Inventive | Inventive | | Inventive | Inventive |
| (1) Euxyl PE | 0.404 g | 0.402 | g | 0.401 g | 0.402 g |
| (2) S68 | 0.602 g | 0.601 | g | 0.603 g | 0.603 g |
| (3) Water | 92.700 g | 90.704 | g | 88.700 g | 86.700 g |
| (4) NaM | 5.001 g | 5.001 | g | 5.000 g | 5.000 g |
| NaM wt % | 5.0% | 5.0% | | 5.0% | 5.0% |
| (A1) X-gum Stock | 0.901 g | 2.701 | g | 4.504 g | 6.302 g |
| (A2) K-gum Stock | 0.104 g | 0.301 | g | 0.501 g | 0.701 g |
| Gum wt % | 0.01% | 0.03% | | 0.05% | 0.07% |
| % X-gum | 90% | 90% | | 90% | 90% |
| (7) Microspheres | 0.302 g | 0.303 | g | 0.302 g | 0.302 g |
| Stability | 1 | 2 | | 2 | 2 |
| Firmness | 8.41N | 10.06N | | 9.97N | 8.03N |
| Temperature | — | 44.6° | C. | — | — |
| Work | — | 915 | J m−3 | — | — |

TABLE 7

| | Sample Y | | Sample Z | | Sample AA | | Sample AB |
|---|---|---|---|---|---|---|---|
| | Inventive | | Comparative | | Inventive | | Inventive |
| (1) Euxyl PE | 0.400 | g | 0.401 | g | 0.401 | g | 0.403 g |
| (2) S68 | 0.600 | g | 0.602 | g | 0.601 | g | 0.602 g |
| (3) Water | 84.703 | g | 92.703 | g | 90.701 | g | 88.701 g |
| (4) NaM | 5.000 | g | 5.000 | g | 5.000 | g | 5.001 g |
| NaM wt % | 5.0% | | 5.0% | | 5.0% | | 5.0% |
| (A1) X-gum Stock | 8.101 | g | 1.001 | g | 3.001 | g | 5.004 g |
| (A2) K-gum Stock | 0.900 | g | — | | — | | — |
| Gum wt % | 0.09% | | 0.01% | | 0.03% | | 0.05% |
| % X-gum | 90% | | 100% | | 100% | | 100% |
| (7) Microspheres | 0.301 | g | 0.300 | g | 0.301 | g | 0.304 g |
| Stability | 2 | | 0 | | 1 | | 1 |
| Firmness | 7.05N | | 10.81N | | 10.54N | | 9.22N |
| Temperature | 37.0° | C. | 43.8° | C. | 42.5° | C. | — |
| Work | 1,810 | J m−3 | 1,145 | J m−3 | 881 | J m−3 | — |

TABLE 8

| | Sample AC | Sample AD | | Sample AE | |
|---|---|---|---|---|---|
| | Inventive | Inventive | | Comparative | |
| (1) Euxyl PE | 0.401 g | 0.401 | g | — | |
| (2) S68 | 0.602 g | 0.602 | g | — | |
| (3) Water | 86.703 g | 84.703 | g | 95.00 | g |
| (4) NaM | 5.000 g | 5.000 | g | 5.00 | g |
| NaM wt % | 5.0% | 5.0% | | 5.0% | |
| (A1) X-gum Stock | 7.003 g | 9.003 | g | — | |
| (A2) K-gum Stock | — | — | | — | |
| Gum wt % | 0.07% | 0.09% | | — | |
| % X-gum | 100% | 100% | | — | |
| (7) Microspheres | 0.301 g | 0.301 | g | 0.303 | g |
| Stability | 1 | 1 | | 0 | |
| Firmness | 9.57N | 9.80N | | 14.31N | |
| Temperature | — | 37.2° | C. | 54.3° | C. |
| Work | — | 840 | J m−3 | 7,730 | J m−3 |

### Example 2

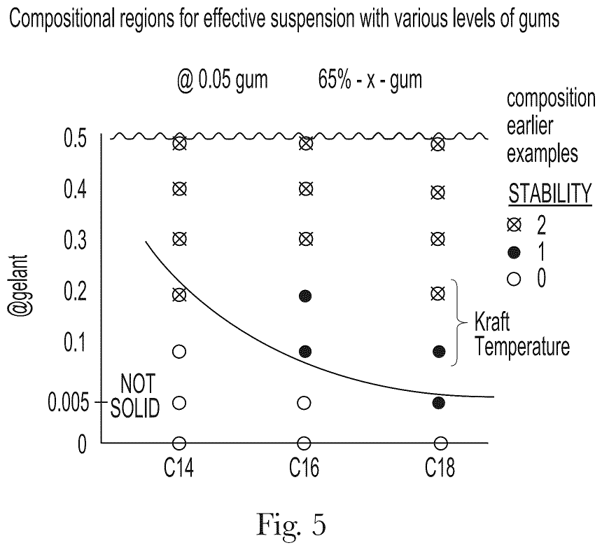

Examples AF-BO use a fixed gum suspension system with different levels and composition of crystallizing agent. The suspension agent is made of 65 wt. % x-gum and 35 wt. % k-gum with a combined 0.05 wt. %, the optimal blend described in Example 1. The composition of the crystallizing agent, sodium myristate, sodium palmitate and sodium stearate, is plotted on the x-axis; the level of crystallizing agent, is plotted on the y-axis (FIG. 5). ‘X’ markers indicate compositions that have a stability grade of ‘0’ as determined by the PHASE STABILITY TEST METHOD, and are comparative compositions; ‘□’ markers indicate compositions that have a stability grade of ‘1’ as determined by the PHASE STABILITY TEST METHOD, and are preferred inventive compositions; ‘O’ markers indicate compositions that have a stability grade of ‘2’ as determined by the PHASE STABILITY TEST METHOD, and are most preferred compositions. Surprisingly, these data demonstrate that the suspension agent can dramatically affect the stability of the composition, where even modest amounts of suspension agent in these examples liquifies the composition, necessitating increases in the level of crystallizing agent to create a stable composition. Equally surprising, the suspension agent affects the shorter chain length crystallizing agent (i.e. sodium myristate) to a greater extent than the longer chain length crystallizing agent (i.e. sodium stearate), as evident by the need for more crystallizing agent in the former. Tables 9-17 also contains firmness (FIRMNESS TEST METHOD), temperature (THERMAL STABILITY TEST METHOD) and work (WATER-EXPRESSION TEST METHOD) data for representative inventive compositions that demonstrate that the prototypes exhibit the desired properties for these rheological solid compositions, even in the presence of the suspension agents.

### Preparation of Compositions

Samples were prepared using a heated mixing device. An overhead mixer (IKA, model RW20 DMZ) and a three-blade impeller design was assembled. All preparations were heated on a heating-pad assembly (VWR, 7×7 CER Hotplate, cat. no. NO97042-690) where heating was controlled with an accompanying probe. All preparations were done in a 250 ml stainless steel beaker (Fischer Scientific).

The NaM/water solution was prepared by first adding the preservatives (1, 2). Water (3), and Na-Myristate (4) were then added to the beaker. The beaker was placed on the heating-pad assembly. The overhead stirrer was placed in the beaker and set to rotate at 100 rpm. The heater was set at 80° C. The preparation was heated to 80° C. The heat was turned off and the preparation was allowed to cool to 60° C.

The final preparation was prepared by adding 1% Xanthan Gum Stock (A1) to the Na-M/Water solution, and the stirring rate was increased to 300-350 rpm. Once the xanthan was completely added and mixed, the 1% Konjac Gum Stock (A2) was added to the Na-M/Water/Xanthan solution, and the stirring rate was increased to 500-550 rpm. Then the solid benefit agents were added to the beaker with continuous stirring and allowed to completely disperse. The composition was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples. Representative data demonstrate that the prototypes exhibit the desired properties for these rheological solid compositions, even in the presence of the suspension agents.

TABLE 9

| | Sample AF | Sample AG | Sample AH | Sample AI | |
|---|---|---|---|---|---|
| | Comparative | Comparative | Inventive | Inventive | |
| (1) Euxyl PE | 0.403 g | 0.400 g | 0.402 g | 0.400 | g |
| (2) S68 | 0.601 g | 0.604 g | 0.602 g | 0.604 | g |
| (3) Water | 93.201 g | 92.700 g | 91.701 g | 90.702 | g |
| (4) NaM | 0.503 g | 1.004 g | 2.001 g | 3.002 | g |
| NaM wt % | 0.50% | 1.00% | 2.00% | 3.0% | |
| (A1) X-gum Stock | 3.253 g | 3.250 g | 3.254 g | 3.254 | g |
| (A2) K-gum Stock | 1.754 g | 1.754 g | 1.753 g | 1.753 | g |
| Gum wt % | 0.05% | 0.05% | 0.05% | 0.05% | |
| % X-gum | 65% | 65% | 65% | 65% | |
| (7) Microspheres | 0.300 g | 0.303 g | 0.304 g | 0.302 | g |
| Stability | 0 | 0 | 2 | 2 | |
| Firmness | NOT SOLID | NOT SOLID | — | 1.65N | |
| Temperature | — | — | — | 38.8° | C. |
| Work | — | — | — | — | |

TABLE 10

| | Sample AJ | | Sample AK | | Sample AL | Sample AM |
|---|---|---|---|---|---|---|
| | Inventive | | Inventive | | Comparative | Comparative |
| (1) Euxyl PE | 0.403 | g | 0.404 | g | — | — |
| (2) S68 | 0.600 | g | 0.603 | g | — | — |
| (3) Water | 89.703 | g | 88.702 | g | 99.501 g | 99.0202 g |
| (4) NaM | 4.003 | g | 5.001 | g | 0.500 g | 1.001 g |
| NaM wt % | 4.00% | | 5.00% | | 0.50% | 1.00% |
| (A1) X-gum Stock | 3.254 | g | 3.252 | g | — | — |
| (A2) K-gum Stock | 1.751 | g | 1.754 | g | — | — |
| Gum wt % | 0.05% | | 0.05% | | — | — |
| % X-gum | 65% | | 100% | | — | — |
| (7) Microspheres | 0.302 | g | 0.300 | g | 0.302 g | 0.301 g |
| Stability | 2 | | 2 | | 0 | 0 |
| Firmness | 6.89N | | 10.29N | | 0.39N | 1.06N |
| Temperature | — | | 39.7° | C. | — | — |
| Work | 543 | J m−3 | 773 | J m−3 | | |

TABLE 11

| | Sample AN | | Sample AO | | Sample AP | | Sample AQ | |
|---|---|---|---|---|---|---|---|---|
| | Comparative | | Comparative | | Comparative | | Comparative | |
| (1) Euxyl PE | — | | — | | — | | — | |
| (2) S68 | — | | — | | — | | — | |
| (3) Water | 98.000 | g | 97.000 | g | 96.002 | g | 95.000 | g |
| (4) NaM | 2.003 | g | 3.002 | g | 4.002 | g | 5.001 | g |
| NaM wt % | 2.00% | | 3.00% | | 3.95% | | 4.94% | |
| (A1) X-gum Stock | — | | — | | — | | — | |
| (A2) K-gum Stock | — | | — | | — | | — | |
| Gum wt % | — | | — | | — | | — | |
| % X-gum | — | | — | | — | | — | |
| (7) Microspheres | 0.301 | g | 0.303 | g | 0.303 | g | 0.303 | g |
| Stability | 0 | | 0 | | 0 | | 0 | |
| Firmness | 3.50N | | 8.60N | | 8.92N | | 15.25N | |
| Temperature | — | | — | | — | | — | |
| Work | 1714 | J m−3 | 2734 | J m−3 | 3365 | J m−3 | 4491 | J m−3 |

TABLE 12

| | Sample AR | Sample AS | Sample AT | | Sample AU | |
|---|---|---|---|---|---|---|
| | Comparative | Inventive | Inventive | | Inventive | |
| (1) Euxyl PE | 0.404 g | 0.401 g | 0.401 | g | 0.400 | g |
| (2) S68 | 0.602 g | 0.602 g | 0.603 | g | 0.600 | g |
| (3) Water | 93.203 g | 92.703 g | 91.704 | g | 90.700 | g |
| (8) NaP | 0.504 g | 1.000 g | 2.000 | g | 3.004 | g |
| NaP wt % | 0.50% | 1.00% | 2.00% | | 3.0% | |
| (A1) X-gum Stock | 3.254 g | 3.252 g | 3.254 | g | 3.253 | g |
| (A2) K-gum Stock | 1.754 g | 1.752 g | 1.750 | g | 1.750 | g |
| Gum wt % | 0.05% | 0.05% | 0.05% | | 0.05% | |
| % X-gum | 65% | 65% | 65% | | 65% | |
| (7) Microspheres | 0.302 g | 0.302 g | 0.302 | g | 0.302 | g |
| Stability | 0 | 1 | 1 | | 2 | |
| Firmness | NOT SOLID | 0.24N | 0.47N | | 0.81N | |
| Temperature | — | — | 48.5° | C. | — | |
| Work | — | — | 156 | J m−3 | 452 | J m−3 |

TABLE 13

| | Sample AV | | Sample AW | | Sample AX | Sample AY | |
|---|---|---|---|---|---|---|---|
| | Inventive | | Inventive | | Comparative | Comparative | |
| (1) Euxyl PE | 0.402 | g | 0.403 | g | — | — | |
| (2) S68 | 0.603 | g | 0.601 | g | — | — | |
| (3) Water | 89.702 | g | 88.704 | g | 99.203 g | 98.702 | g |
| (8) NaP | 4.000 | g | 5.000 | g | 0.503 g | 1.000 | g |
| NaP wt % | 4.00% | | 5.00% | | 0.50% | 1.0% | |
| (A1) X-gum Stock | 3.250 | g | 3.250 | g | — | — | |
| (A2) K-gum Stock | 1.753 | g | 1.751 | g | — | — | |
| Gum wt % | 0.05% | | 0.05% | | — | — | |
| % X-gum | 65% | | 65% | | — | — | |
| (7) Microspheres | 0.303 | g | 0.300 | g | 0.300 g | 0.300 | g |
| Stability | 2 | | 2 | | 0 | 0 | |
| Firmness | 1.61N | | 2.66N | | 0.18N | 0.22N | |
| Temperature | — | | — | | — | — | |
| Work | 979 | J m−3 | 444 | J m−3 | SOFT | 406 | J m−3 |

TABLE 14

| | Sample AZ | | Sample BA | | Sample BB | | Sample BC | |
|---|---|---|---|---|---|---|---|---|
| | Comparative | | Comparative | | Comparative | | Comparative | |
| (1) Euxyl PE | — | | — | | — | | — | |
| (2) S68 | — | | — | | — | | — | |
| (3) Water | 97.703 | g | 96.703 | g | 95.700 | g | 94.701 | g |
| (8) NaP | 2.003 | g | 3.000 | g | 4.001 | g | 5.000 | g |
| NaP wt % | 2.00% | | 3.00% | | 4.00% | | 5.0% | |
| (A1) X-gum Stock | — | | — | | — | | — | |
| (A2) K-gum Stock | — | | — | | — | | — | |
| Gum wt % | — | | — | | — | | — | |
| % X-gum | — | | — | | — | | — | |
| (7) Microspheres | 0.301 | g | 0.301 | g | 0.302 | g | 0.300 | g |
| Stability | 0 | | 0 | | 0 | | 0 | |
| Firmness | 0.44N | | 0.79N | | 1.40N | | 2.54N | |
| Temperature | — | | — | | — | | — | |
| Work | 605 | J m−3 | 1159 | J m−3 | 2468 | J m−3 | 2910 | J m−3 |

TABLE 15

| | Sample BD | Sample BE | Sample BF | | Sample BG | |
|---|---|---|---|---|---|---|
| | Comparative | Inventive | Inventive | | Inventive | |
| (1) Euxyl PE | 0.402 g | 0.402 g | 0.400 | g | 0.402 | g |
| (2) S68 | 0.603 g | 0.601 g | 0.604 | g | 0.604 | g |
| (3) Water | 93.203 g | 92.703 g | 91.704 | g | 89.702 | g |
| (9) NaS | 0.500 g | 1.002 g | 2.003 | g | 3.002 | g |
| NaS wt % | 0.50% | 1.00% | 2.00% | | 3.0% | |
| (A1) X-gum Stock | 3.252 g | 3.251 g | 3.253 | g | 3.254 | g |
| (A2) K-gum Stock | 1.754 g | 1.753 g | 1.754 | g | 1.753 | g |
| Gum wt % | 0.05% | 0.05% | 0.05% | | 0.05% | |
| % X-gum | 65% | 65% | 65% | | 65% | |
| (7) Microspheres | 0.303 g | 0.300 g | 0.300 | g | 0.300 | g |
| Stability | 1 | 1 | 2 | | 2 | |
| Firmness | NOT SOLID | 0.09N | 0.58N | | 0.96N | |
| Temperature | — | — | — | | 58.2° | C. |
| Work | | SOFT | 379 | J m−3 | 668 | J m−3 |

TABLE 16

| | Sample BH | Sample BI | | Sample BJ | Sample BK | |
|---|---|---|---|---|---|---|
| | Inventive | Inventive | | Comparative | Comparative | |
| (1) Euxyl PE | 0.401 g | 0.401 | g | — | — | |
| (2) S68 | 0.603 g | 0.604 | g | — | — | |
| (3) Water | 88.700 g | 88.703 | g | 99.203 g | 98.701 | g |
| (9) NaS | 4.002 g | 5.000 | g | 0.502 g | 1.002 | g |
| NaS wt % | 4.00% | 5.00% | | 0.50% | 1.0% | |
| (A1) X-gum Stock | 3.253 g | 3.253 | g | — | — | |
| (A2) K-gum Stock | 1.753 g | 1.753 | g | — | — | |
| Gum wt % | 0.05% | 0.05% | | — | — | |
| % X-gum | 65% | 65% | | — | — | |
| (7) Microspheres | 0.301 g | 0.300 | g | 0.302 g | 0.300 | g |
| Stability | 2 | 2 | | 0 | 0 | |
| Firmness | — | — | | 0.16N | 0.18N | |
| Temperature | — | 62.5° | C. | — | — | |
| Work | | | | SOFT | 395 | J m−3 |

TABLE 17

| | Sample BL | | Sample BM | | Sample BN | | Sample BO | |
|---|---|---|---|---|---|---|---|---|
| | Comparative | | Comparative | | Comparative | | Comparative | |
| (1) Euxyl PE | — | | — | | — | | — | |
| (2) S68 | — | | — | | — | | — | |
| (3) Water | 97.701 | g | 96.701 | g | 95.700 | g | 94.701 | g |
| (9) NaS | 2.000 | g | 3.00 | g | 4.001 | g | 5.000 | g |
| NaS wt % | 2.00% | | 3.00% | | 4.00% | | 1.0% | |
| (A1) X-gum Stock | — | | — | | — | | — | |
| (A2) K-gum Stock | — | | — | | — | | — | |
| Gum wt % | — | | — | | — | | — | |
| % X-gum | — | | — | | — | | — | |
| (7) Microspheres | 0.302 | g | 0.301 | g | 0.303 | g | 0.300 | g |
| Stability | 0 | | 0 | | 0 | | 0 | |
| Firmness | 0.45N | | 0.71N | | 1.07N | | 1.36N | |
| Temperature | — | | — | | — | | — | |
| Work | 1001 | J m−3 | 657 | J m−3 | 2261 | J m−3 | 1643 | J m−3 |

### Example 3

This example demonstrates compositions effective at suspending perfume capsules (PC)—considered a proxy for insoluble encapsulated active agent, using the suspension agents described in FIG. 4 and FIG. 5. Perfume capsules have an oil core surrounded by a thin solid shell. Not wishing to be bound by theory, because the perfume is less dense than the aqueous phase, the capsules will float to the top of the composition in the absence of suspension agents. The inventive Sample (Sample BP) with a suspension agent and was shown to have stability grade of ‘2’ as determined by the PHASE STABILITY TEST METHOD while the comparative Sample (Sample BQ) without a suspension agent was shown to have stability grade of ‘0’ as determined by the PHASE STABILITY TEST METHOD.

### Preparation of Compositions

The inventive composition was prepared by adding Euxyl PE 9010 (1), Symdiol 68 (2), water (3), and sodium myristate (4) to a stainless-steel beaker (Beaker Griffin 250 mL Stainless Steel Beaker, VWR Catalog: 74360-008, or equivalent). The beaker was placed on the heating-pad assembly (VWR Hotplate with Thermocouple, SN: 160809002) and the overhead stirrer (IKA RW20DZM.n Overhead mixer, SN: 03.153609) was placed into the beaker and set to rotate at 100 rpm. The heater was set at 80° C. The preparation was heated to 80° C. Once the solution reached 80° C. the solution was cooled down to 60° C., at which time the x-gum (A1) and k-gum (A2) solutions were added along with the PC (13). The mixer was increased by 100 rpm for each ingredient added. The solution was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples. Representative data demonstrates that the prototypes exhibit the desired properties for these rheological solid compositions, even in the presence of the suspension agents.

The comparative compositions were prepared by adding Euxyl PE 9010 (1), Symdiol 68 (2), water (3), and sodium myristate (4) to a stainless-steel beaker (Beaker Griffin 250 mL Stainless Steel Beaker, VWR Catalog: 74360-008, or equivalent). The beaker was placed on the heating-pad assembly (VWR Hotplate with Thermocouple, SN: 160809002) and the overhead stirrer (IKA RW20DZM.n Overhead mixer, SN: 03.153609) was placed into the beaker and set to rotate at 100 rpm. The heater was set at 80° C. The preparation was heated to 80° C. Once the solution reached 80° C. the solution was cooled down to 60° C., at which time the PC (13) were added. The mixer was increased by 100 rpm for each ingredient added. The solution was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples.

TABLE 18

| | |
|---|---|
| Sample BP | Sample BQ |
| Inventive | Comparative |

| | | | | |
|---|---|---|---|---|
| (1) Euxyl PE | 0.400 | g | 0.400 | g |
| (2) S68 | 0.603 | g | 0.603 | g |
| (3) Water | 87.004 | g | 92.001 | g |
| (4) NaM | 5.000 | g | 5.002 | g |

| | | |
|---|---|---|
| NaM wt % | 5.00% | 5.00% |

| | | | |
|---|---|---|---|
| (A1) X-gum Stock | 3.252 | g | — |
| (A2) K-gum Stock | 1.752 | g | — |

| | | |
|---|---|---|
| Gum wt % | 0.05% | — |
| % X-gum | 65% | — |

| | | | | |
|---|---|---|---|---|
| (13) PC | 2.004 | g | 2.003 | g |

| | | |
|---|---|---|
| Stability | 2 | 0 |
| Firmness | 8.7N | 9.0N |

| | | | | |
|---|---|---|---|---|
| Temperature | 36.8° | C. | 38.5° | C. |
| Work | 1425 | J m−3 | 994 | J m−3 |

### Example 4

This example demonstrates compositions effective at suspending starch, considered a proxy for insoluble active particles that sediment, using the suspension agents described in FIG. 4 and FIG. 5. The starch was added to give a silky-smooth feel to the skin and surfaces. Not wishing to be bound by theory, since starch is both denser than the aqueous phase and insoluble it will settle in the aqueous phase. The inventive Sample (Sample BR) with the suspension agent and was shown to have stability grade of ‘2’ as determined by the PHASE STABILITY TEST METHOD while the comparative Sample (Sample BS) without the suspension agent was shown to have stability grade of ‘0’ as determined by the PHASE STABILITY TEST METHOD.

### Preparation of Compositions

The inventive sample was prepared by adding Euxyl PE 9010 (1), Symdiol 68 (2), water (3), and sodium myristate (4) to a stainless-steel beaker (Beaker Griffin 250 mL Stainless Steel Beaker, VWR Catalog: 74360-008, or equivalent). The beaker was placed on the heating-pad assembly (VWR Hotplate with Thermocouple, SN: 160809002) and the overhead stirrer (IKA RW20DZM.n Overhead mixer, SN: 03.153609) was placed into the beaker and set to rotate at 100 rpm. The heater was set at 80° C. The preparation was heated to 80° C. Once the solution reached 80° C., the solution was cooled down to 60° C., at which time the gums X-gum (A1) and K-gum (A2) solutions were added along with the starch (10). The mixer was increased by 100 rpm for each ingredient added. The composition was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples. Representative data demonstrate that the prototypes exhibit the desired properties for these rheological solid compositions, even in the presence of the suspension agents.

The comparative sample was prepared by adding Euxyl PE 9010 (1), Symdiol 68 (2), water (3), and sodium myristate (4) to a stainless-steel beaker (Beaker Griffin 250 mL Stainless Steel Beaker, VWR Catalog: 74360-008, or equivalent). The beaker was placed on the heating-pad assembly (VWR Hotplate with Thermocouple, SN: 160809002) and the overhead stirrer (IKA RW20DZM.n Overhead mixer, SN: 03.153609) was placed into the beaker and set to rotate at 100 rpm. The heater was set at 80° C. The preparation was heated to 80° C. Once the solution reached 80° C., the solution was cooled down to 60° C., at which time the starch (10) was added. The mixer was increased by 100 rpm for each ingredient added. The composition was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples.

TABLE 19

| | |
|---|---|
| Sample BR | Sample BS |
| Inventive | Comparative |

| | | | | |
|---|---|---|---|---|
| (1) Euxyl PE | 0.403 | g | 0.400 | g |
| (2) S68 | 0.601 | g | 0.604 | g |
| (3) Water | 87.003 | g | 92.002 | g |
| (4) NaM | 5.002 | g | 5.000 | g |

| | | |
|---|---|---|
| NaM wt % | 5.0% | 5.0% |

| | | | |
|---|---|---|---|
| (A1) X-gum Stock | 3.252 | g | — |
| (A2) K-gum Stock | 1.752 | g | — |

| | | |
|---|---|---|
| Gum wt % | 0.05% | — |
| % X-gum | 65% | — |

| | | | | |
|---|---|---|---|---|
| (10) Starch | 2.003 | g | 2.000 | g |

| | | |
|---|---|---|
| Stability | 2 | 0 |
| Firmness | 6.8N | 10.5N |

| | | | | |
|---|---|---|---|---|
| Temperature | 33.6° | C. | 34.8° | C. |
| Work | 664 | J m−3 | 263 | J m−3 |

### Example 5

This example demonstrates compositions effective at suspending coconut oils, considered a proxy for liquid-to-solid insoluble active agents, using the suspension agents described in FIG. 4 and FIG. 5. Coconut oils are used as an emollient on skin and hair. During the process of making these compositions, the coconut oil melts into a liquid and is then emulsified in the stirred composition. Upon cooling, the oils harden into solid particles. Not wishing to be bound by theory, since the oil is less dense than the composition it will float to the top of the mixture in the absence of a suspension agent. The inventive Sample (Sample BT) with the suspension agent and was shown to have stability grade of ‘2’ as determined by the PHASE STABILITY TEST METHOD while the comparative Sample (Sample BU) without the suspension agent and was shown to have stability grade of ‘0’ as determined by the PHASE STABILITY TEST METHOD.

### Preparation of Compositions

The inventive sample was prepared by adding Euxyl PE 9010 (1), Symdiol 68 (2), water (3) and sodium myristate (4) to a stainless-steel beaker (Beaker Griffin 250 mL Stainless Steel Beaker, VWR Catalog: 74360-008, or equivalent). The beaker was placed on the heating-pad assembly (VWR Hotplate with Thermocouple, SN: 160809002) and the overhead stirrer (IKA RW20DZM.n Overhead mixer, SN: 03.153609) was placed into the beaker and set to rotate at 100 rpm. The heater was set at 80° C. The preparation was heated to 80° C. Once the solution reached 80° C., the solution was cooled down to 60° C., at which time the x-gum (A1) and k-gum (A2) solutions were added along with the coconut oil (12). The mixer was increased by 100 rpm for each ingredient added. The composition was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples. Representative data demonstrate that the prototypes exhibit the desired properties for these rheological solid compositions, even in the presence of the suspension agents.

The comparative sample was prepared by adding Euxyl PE 9010 (1), Symdiol 68 (2), water (3), and sodium myristate (4) to a stainless-steel beaker (Beaker Griffin 250 mL Stainless Steel Beaker, VWR Catalog: 74360-008, or equivalent). The beaker was placed on the heating-pad assembly (VWR Hotplate with Thermocouple, SN: 160809002) and the overhead stirrer (IKA RW20DZM.n Overhead mixer, SN: 03.153609) was placed into the beaker and set to rotate at 100 rpm. The heater was set at 80° C. The preparation was heated to 80° C. Once the solution reached 80° C., the composition was cooled down to 60° C., at which time the coconut oil (12) was added. The mixer was increased by 100 rpm for each ingredient added. The composition was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples.

TABLE 20

| | |
|---|---|
| Sample BT | Sample BU |
| Inventive | Comparative |

| | | | | |
|---|---|---|---|---|
| (1) Euxyl PE | 0.403 | g | 0.400 | g |
| (2) S68 | 0.601 | g | 0.604 | g |
| (3) Water | 87.003 | g | 92.000 | g |
| (4) NaM | 5.002 | g | 5.000 | g |

| | | |
|---|---|---|
| NaM wt % | 5.00% | 5.00% |

| | | | |
|---|---|---|---|
| (A1) X-gum Stock | 3.252 | g | — |
| (A2) K-gum Stock | 1.752 | g | — |

| | | |
|---|---|---|
| Gum wt % | 0.05% | — |
| % X-gum | 65% | — |

| | | | | |
|---|---|---|---|---|
| (13) PC | 2.003 | g | 2.000 | g |

| | | |
|---|---|---|
| Stability | 2 | 0 |
| Firmness | 8.7N | 9.0N |

| | | | | |
|---|---|---|---|---|
| Temperature | 39.7° | C. | 39.9° | C. |
| Work | 1368 | J m−3 | 1432 | J m−3 |

### Example 6

This example demonstrates compositions effective at suspending peppermint oils, considered a proxy for liquid insoluble active agents, using the suspension agents described in FIG. 4 and FIG. 5. Peppermint oils are natural or essential oils used to naturally treat skin and hair. This oil remains liquid throughout the entire preparation process. Not wish to be bound by theory, since it is less dense than the aqueous phase it will float to the top of the composition in the absence of a suspension agent. Surprisingly, these oils also ‘interfere’ with the crystallization process of the crystallizing agent, the level of which needs to be adjusted for the presence of the oils. The inventive examples with the suspension agent was shown to have stability grade of ‘2’ as determined by the PHASE STABILITY TEST METHOD (Samples BV and BX) while the comparative example without the suspension agent (Sample BZ) has a stability grade of ‘0’ as determined by the PHASE STABILITY TEST METHOD. Sample BY comprising the suspension agent shows a stability grade of ‘0’ as determined by the PHASE STABILITY TEST METHOD due to the high amount of peppermint oil, which resulted in failure in stability and firmness.

### Preparation of Compositions

The inventive sample was prepared by adding Euxyl PE 9010 (1), Symdiol 68 (2), water (3), and sodium myristate (4) to a stainless-steel beaker (Beaker Griffin 250 mL Stainless Steel Beaker, VWR Catalog: 74360-008, or equivalent). The beaker was placed on the heating-pad assembly (VWR Hotplate with Thermocouple, SN: 160809002) and the overhead stirrer (IKA RW20DZM.n Overhead mixer, SN: 03.153609) was placed into the beaker and set to rotate at 100 rpm. The heater was set at 80° C. The preparation was heated to 80° C. Once the solution reached 80° C., the solution was cooled down to 60° C., at which time the x-gum (A1) and k-gum (A2) solutions were added along with the peppermint oil (11). The mixer was increased by 100 rpm for each ingredient added. The composition was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples. Representative data demonstrate that the prototypes exhibit the desired properties for these rheological solid compositions, even in the presence of the suspension agents. Representative data demonstrate that the prototypes exhibit the desired properties for these rheological solid compositions, even in the presence of the suspension agents.

The comparative sample was prepared by adding Euxyl PE 9010 (1), Symdiol 68 (2), water (3), and sodium myristate (4) to a stainless-steel beaker (VWR Hotplate with Thermocouple, SN: 160809002). The beaker was placed on the heating-pad assembly (DETAILS) and the overhead stirrer (IKA RW20DZM.n Overhead mixer, SN: 03.153609) was placed into the beaker and set to rotate at 100 rpm. The heater was set at 80° C. The preparation was heated to 80° C. Once the solution reached 80° C., the solution was cooled down to 60° C., at which time the peppermint oil (11) was added. The mixer was increased by 100 rpm for each ingredient added. The composition was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples.

TABLE 21

| | Sample BV | | Sample BX | | Sample BY | | Sample BZ |
|---|---|---|---|---|---|---|---|
| | Inventive | | Inventive | | Comparative | | Comparative |
| (1) Euxyl PE | 0.40 | g | 0.40 | g | 0.40 | g | 0.40 g |
| (2) S68 | 0.60 | g | 0.60 | g | 0.60 | g | 0.06 g |
| (3) Water | 88.75 | g | 88.50 | g | 88.00 | g | 93.75 g |
| (4) NaM | 5.00 | g | 5.00 | g | 5.00 | g | 5.00 g |
| NaM wt % | 5.0% | | 5.0% | | 5.0% | | 5.0% |
| (A1) X-gum Stock | 3.25 | g | 3.25 | g | 3.25 | g | — |
| (A2) K-gum Stock | 1.75 | g | 1.75 | g | 1.75 | g | — |
| Gum wt % | 0.05% | | 0.05% | | 0.05% | | — |
| % X-gum | 65.0% | | 65.0% | | 65.0% | | — |
| (11) Peppermint | 0.25 | g | 0.50 | g | 1.00 | g | 0.25 g |
| Stability | 2 | | 2 | | 0 | | 0 |
| Firmness | 7.2N | | 4.9N | | NOT SOLID | | — |
| Temperature | 37.3° | C. | 35.8° | C. | — | | — |
| Work | 371 | J m−3 | 640 | J m−3 | 264 | J m−3 | — |

### Example 7

This example demonstrates that it is possible to create stable compositions with a large weight amount of a very complex mixture of insoluble active agents, sometimes with modifications of the composition. All compositions contain about 10 wt. % of insoluble active agents and all compositions contain a blend of seven different oils (see Oil Blend). One skilled in the art recognizes this as a very large level of dispersed insoluble active agent. Samples CA, CB and CC utilizing 0.09 wt. % of a x-gum and k-gum blend suspension agent system (see Example 1). As previously noted, some oils require adjustment in the amount of the crystallizing agent. In this example, it is increased to about 5 wt. % to compensate for the weakening effect associated with the presence of the oils. Sample CA still has too small an amount of suspension agent to stabilize the composition relative to previous examples which have 0.3-2.0 wt. % insoluble active agent particles. In Samples CB and CC NaCl is increased to raise the thermal stability of the composition so that crystallization agents crystallize faster than otherwise. Comparative sample CD omits the suspension agent which results in nearly complete separation of the oils in the form of a thick layer on top of the composition, rendering it unfit for consumer use.

### (A3) Preparation of Oil Blend

The following ingredients were weighed and added to a 1 liter beaker: L-Menthol (14), Nutmeg Oil (15), Camphor (16), Eucalyptus Oil (17), Cedar Leaf Oil (18), Turpentine Containing Antioxidant (19), Thymol NF (20). They were mixed using an overhead mixer device rotating at 100 rpm until the solution was completely clear and then mixed for an additional 10 minutes.

### Preparation of Compositions

Deionized water (3) was added to a 16 oz wide mouth glass jar (VWR, Cat #: glc-01700). Sodium chloride (21) was added to the jar. The jar was swirled until the sodium chloride was completely dissolved. It was then placed in a 90° C.-controlled water bath (Insta-therm 2600 mL, controlled by Staco INC Variable autotransformer) and the mixture was brought to bath temperature. A large magnetic stir bar was added to the jar and spun at 200 rpm. Sodium palmitate (8) was added to the jar. It was loosely capped to prevent water loss and to prevent pressurization. The mixture was stirred until the sodium palmitate completely dissolved. The jar was removed from the bath and placed in a second 80° C.-controlled water bath (VWR 7×7 Stir PRO w/Temp probe). The first lid was replaced with a second lid containing two, 8 mm holes: one hole was in the center to accommodate the impeller shaft and one hole offset half-way between the edge and the center of the jar to allow addition of the remaining ingredients. A 4-blade impeller was installed by passing the shaft through the center hole in the lid and placing the blade into the mixture when fastening the lid. The impeller was set to spin at 450 rpm (Caframo BDC 3030). Euxyl PE (1) and Symdiol 68 (2) were added through the second hole in the lid and x-gum (A1) and k-gum (A2) stock solutions were added dropwise using a 1ml positive displacement syringe also through the second hole. After mixing for a minute, the oil blend (A3) was added through the same hole. The impeller speed was increased to 750 rpm for two additional minutes. The final mixture was poured into 60 ml cups (Flak-Tech, Max 60 Cup Translucent, Cat #501 222t), to cool and crystallize. Firmness measurements were made with the FIRMNESS TEST METHOD and thermal stability measurements were made by the THERMAL STABILITY TEST METHOD on the 50 ml sample; water-expression measurements were made by the WATER-EXPRESSION TEST METHOD on the two 25 ml samples

TABLE 22

| | Sample CA | | Sample CB | | Sample CC | | Sample CD |
|---|---|---|---|---|---|---|---|
| | Comparative | | Inventive | | Inventive | | Comparative |
| (3) Water | 75.21 | g | 71.80 | g | 70.43 | g | 84.12 g |
| (21) NaCl | — | | 3.51 | g | 2.71 | g | — |
| (8) NaP | 5.08 | g | 5.02 | g | 7.02 | g | 5.00 g |
| NaP wt % | 5% | | 5% | | 7% | | 5% |
| (1) Euxyl PE | 0.36 | g | 0.36 | g | 0.36 | g | 0.36 g |
| (2) S68 | 0.54 | g | 0.54 | g | 0.54 | g | 0.54 g |
| (A1) X-gum | 3.57 | g | 3.61 | g | 3.75 | g | — |
| (A2) K-gum | 5.52 | g | 5.49 | g | 5.44 | g | — |
| Gum wt % | 0.09% | | 0.09% | | 0.09% | | — |
| % X-gum | 40.5% | | 39.5% | | 44% | | — |
| (A3) Oil blend | 9.85 | g | 9.97 | g | 10.00 | g | 10.02 |
| Stability | 0 | | 1 | | 2 | | 0 |
| Firmness | 1.9N | | 2.9N | | 5.1N | | — |
| Temperature | 33.3° | C. | 43.6° | C. | 42.6° | C. | — |

| | | | | |
|---|---|---|---|---|
| Work | — | — | — | — |

### Example 8

This example demonstrates that it is possible to create stable compositions with a large weight amount of a very complex mixtures of insoluble active agents, by increasing the amount of suspending agent. All compositions contain about 10 wt. %-12 wt% of insoluble active agents and all compositions contain a blend of six different oils (Sample CF) and petrolatum (Sample CE) (see Petrolatum/Oil Blend), with x-gum as a suspension agent at elevated concentrations. Having a higher concentration of x-gum is particularly important since the petrolatum is liquid at process temperatures and converts to a solid at room temperature. Each composition uses about 0.30 wt. % of x-gum as the suspension agent. This is a significantly higher concentration than when x-gum and k-gum are combined as a mixture in EXAMPLE 1 and highlighted in EXAMPLE 7. Not wishing to be bound by theory, in contrast to the gum blends, the x-gum alone increases the viscosity of the composition before the formation of the mesh. Furthermore, the amount of the crystallizing agent is increased to about 5 wt. % to compensate for the weakening effect associated with the presence of the oils in the composition. The higher level of suspension agent allows for greater stability.

### (A4) Preparation of X-gum Stock in Glycerol

The x-gum stock was prepared by adding 9.001 grams of glycerol (9) to 60 ml Speed Mixer Cup (Flak-Tech, Max 60 Cup Translucent Reorder Number: 501 222t). 1.007 grams of x-gum (5) were added to the cup. It was placed in the Speed Mixer (Flacktek, Inc.) and run at 3500 rpm for one minute. The mixture was allowed to sit quiescently for an hour at which point is was re-mixed at 3500 rpm for another 10 seconds.

### (A5) Preparation of Oil Blend

The following were weighed and added to a 1 liter beaker: L-Menthol (14), Nutmeg Oil (15), Camphor (16), Eucalyptus Oil (17), Cedar Leaf Oil (18), Thymol NF (20). They were mixed using an overhead impeller mixing device at 100 rpm until the solution was completely clear, then mixed for an additional 10 minutes.

### (A6) Petrolatum/Oil Blend

10.227 g of the oil mixture (A5) was pre-heated with 14.02 g petrolatum (22) in a glass vial to 40° C. on the hotplate (VWR digital heat block, Cat. Number 12621-088). It was then vortexed for 10 seconds at max speed and returned to the 40° C. hotplate for no longer than 60 minutes before being used to prepare the example compositions.

### Preparation of Compositions

Deionized water (3) was added to a 16 oz wide mouth glass jar (VWR). Sodium chloride (21) was added to the jar. The jar was swirled until the salt completely dissolved. It was then placed in a 90° C.-controlled water bath (Insta-therm 2600 mL, controlled by Staco INC Variable autotransformer) and the mixture was brought to bath temperature. A large magnetic stir bar was added to the jar and spun at 200 rpm. Sodium palmitate (8) was added to the jar. It was loosely capped to prevent water loss but also prevent pressurization. The mixture was stirred until the sodium palmitate completely dissolved. The jar was removed from the bath and placed in a second 80° C.-controlled water bath (VWR 7×7 Stir PRO w/Temp probe). The first lid was replaced with a second lid containing two, 8 mm holes: one hole was in the center to accommodate the impeller shaft and one hole offset half-way between the edge and the center of the jar to allow addition of the remaining ingredients. A 4-blade impeller was installed by passing the shaft through the center hole in the lid and placing the blade into the mixture when fasting the lid. The impeller was set to spin at 450 rpm (Caframo BDC 3030). Then, Euxyl PE (1) and Symdiol 68 (2) were added through the second hole in the lid. x-gum-in-glycerol stock solution (A4) was added dropwise using a 1 ml positive displacement syringe also through the second hole. After mixing for a minute, the oil/petrolatum blend (A6) was added through the same hole. The impeller speed was increased to 750 rpm for two additional minutes. The final mixture was poured into 60 ml cups (Flak-Tech, Max 60 Cup Translucent Reorder Number: 501 222t) to cool and crystallize. Firmness measurements were made with the FIRMNESS TEST METHOD and thermal stability measurements were made by the THERMAL STABILITY TEST METHOD on the 50 ml sample; water-expression measurements were made by the WATER-EXPRESSION TEST METHOD on the two 25 ml samples. Representative data demonstrate that the prototypes exhibit the desired properties for these rheological solid compositions, even in the presence of the suspension agents.

TABLE 23

| | |
|---|---|
| Sample CE | Sample CF |
| Inventive | Inventive |

| | | | | |
|---|---|---|---|---|
| (3) Water | 76.41 | g | 77.61 | g |
| (21) NaCl | 3.51 | g | 3.51 | g |
| (8) NaP | 5.01 | g | 5.01 | g |

| | | |
|---|---|---|
| NaP wt % | 5.0% | 5.0% |

| | | | | |
|---|---|---|---|---|
| (1) Euxyl PE | 0.10 | g | 0.36 | g |
| (2) S68 | 0.00 | g | 0.54 | g |
| (A4) X-gum Stock | 2.99 | g | 3.05 | g |

| Gum wt % | 0.30% | 0.30% | |
|---|---|---|---|
| % X-gum | 100% | 100% | |
| (A5) Oil Blend | — | 10.04 | g |

| | | | |
|---|---|---|---|
| (A6) Petrolatum/Oil Blend | 12.09 | g | — |

| | | |
|---|---|---|
| Stability | 2 | 1 |
| Firmness | — | 4.8N |

| | | | | |
|---|---|---|---|---|
| Temperature | 51.0° | C. | 43.0° | C. |

| | | |
|---|---|---|
| Work | — | — |

### Example 9

These samples demonstrate that it is possible to create inventive compositions that have a large weight percent of a very complex mixture of insoluble active agents with about 10 wt. % of a blend of seven different oils and petrolatum (Samples CG and CH), using microfibers as a suspension agent. Not wishing to be bound by theory, it is believed that the microfibers increase the viscosity of the composition before the formation of the mesh. Without sodium chloride (Sample CG) or with the sodium chloride (CH), to raise the thermal stability of the composition so that crystallization agents crystallize faster than otherwise, both compositions are stable. The microfibers upwards of 0.2 wt. %-0.27 wt. % are effective at suspending the insoluble active agent, similar to EXAMPLE 7.

### (A7) Petrolatum/Oil Blend

10.227 g of the oil mixture (A5) was pre-heated with 14.02 g petrolatum (22) in a glass vial to 40° C. on the hotplate (VWR digital heat block, Cat. Number 12621-088). The vial is then vortexed for 10 seconds at max speed and returned to 40° C. hotplate for no longer than 60 minutes before being used to prepare the example compositions.

### Preparation of Compositions

Deionized water (3) was added to a 16 oz wide mouth glass jar (VWR). The Rheocrysta c-2sp solution (24) was added dropwise using a 1 ml positive displacement syringe. Sodium chloride (21) was added to the jar. The jar was swirled until the salt completely dissolved. It was then placed in a 90° C.-controlled water bath (Insta-therm 2600 mL, controlled by Staco INC Variable autotransformer) and the mixture was brought to bath temperature. A large magnetic stir bar was added to the jar and spun at 200 rpm. Sodium palmitate (8) was added to the jar. It was loosely capped to prevent water loss but also prevent pressurization. The mixture was stirred until the sodium palmitate completely dissolved. The jar was removed from the bath and placed in a second 80° C.-controlled water bath (VWR 7×7 Stir PRO w/Temp probe). The first lid was replaced with a second lid containing two, 8 mm holes: one hole was in the center set for the impeller shaft and one hole offset halfway between the edge and the center of the jar set for adding the remaining ingredients. A 4-blade impeller was installed by passing the shaft through the center hole in the lid and placing the blade into the mixture when fasting the lid. The impeller was set to spin at 450 rpm (Caframo BDC 3030). Then, Euxyl PE (1) and Symdiol 68 (2) were added through the second hole in the lid. After mixing for a minute, the oil/petrolatum blend (A7) or oil mixture (A3) was added through the same hole. The impeller speed was increased to 750 rpm for two additional minutes. The final mixture was poured into 60 ml cups (Flak-Tech, Max 60 Cup Translucent Reorder Number: 501 222t) to cool and crystallize.

TABLE 24

| | Sample CG | Sample CH | |
|---|---|---|---|
| | Inventive | Inventive | |
| (3) Water | 69.50 g | 70.65 | g |
| (24) Rheo solution | 13.45 g | 10.27 | g |
| % Suspension Agent | 0.27% | 0.20% | |
| (21) NaCl | — | 3.50 | g |
| (8) NaP | 5.01 g | 5.01 | g |
| NaP wt % | 5% | 5% | |
| (1) Euxyl PE | — | 0.36 | g |
| (2) S68 | — | 0.54 | g |
| (A7) Petrolatum/Oil Blend | 12.03 g | — | |
| (A3) Oil Mixture | — | 10.03 | g |

| | | |
|---|---|---|
| Stability | 1 | 1 |
| Firmness | — | — |
| Temperature | — | — |
| Work | — | — |

### Example 10

These samples demonstrate that it is possible to create inventive compositions that contain a large weight percent of a very complex mixtures of insoluble active agents that have about 10 wt. % of a blend of seven different oils and petrolatum (Samples CI and CJ), using laponite clay as a suspension agent. Not wishing to be bound by theory, it is believed that electrostatic attractions between laponite clay particles create a house-of-card structure that creates a yield stress in the composition before the formation of the mesh. As with EXAMPLE 8 and EXAMPLE 9, the higher level of suspension agent may create stable compositions (Sample CI). Surprisingly, the addition of sodium chloride (Sample CJ) results in unstable product, in contrast to previous EXAMPLES 7-9. In this case, one skilled in the art recognizes that adding sodium chloride eliminates the electrostatic attractions between laponite clay particles, the house-of-card structure does not form.

### (A8) Preparation of Laponite Solution

Prepare a 5% Laponite XLG stock using 2.500 g Laponite XLG (c4039229), and 47.512 g DI water, speed mixing at 3500 rpm for 1 minute, and allowed to rest overnight. Then the water is added to the jar. The laponite stock solution is then added, and is stirred into solution using a Q line stirrer model 134:1 set to 25 on the dial with a 4 blade impeller. The salt is then added in. Then, the jar is capped. It is then placed in the 90° C. water bath and the sodium palmitate is added, and it is stirred using a stir bar in the water bath until a cloudy homogenous solution. It is then placed in an 80° C. secondary container.

### (A9) Petrolatum/Oil Blend

The following were weighed and added to a 1 liter beaker: L-Menthol (14); Nutmeg Oil (15); Camphor (16); Eucalyptus Oil (17); Cedar Leaf Oil (18); Thymol (20). 10.227 g of this oil mixture and 14.02 g petrolatum (22) were heated to 40° C. in a glass vial on the hotplate (VWR digital heat block, Cat. Number 12621-088). The vial is then vortexed for 10 seconds at max speed and returned to the 40° C. hotplate for no longer than 60 minutes before being used to prepare the example compositions.

### (A10) Petrolatum/Oil Blend

5.040 g of the oil mixture (A5) and 5.046 g petrolatum (22) were heated to 40° C. in a glass vial on the hotplate (VWR digital heat block, Cat. Number 12621-088). The vial is then vortexed for 10 seconds at max speed, and returned to the 40° C. hotplate for no longer than 60 minutes, before being used to prepare the example compositions.

### Preparation of Compositions

Deionized water (3) was added to a 16 oz wide mouth glass jar (VWR). The Laponite solution (25) was added dropwise using a 1 ml positive displacement syringe also through the second hole, and mixed for another minute. Sodium chloride (21) was added to the jar. The jar was swirled until the salt completely dissolved. It was then placed in a 90° C.-controlled water bath (Insta-therm 2600 mL, controlled by Staco INC Variable autotransformer) and the mixture was brought to bath temperature. A large magnetic stir bar was added to the jar and spun at 200 rpm. Sodium palmitate (8) was added to the jar. It was loosely capped to prevent water loss but also prevent pressurization. The mixture was stirred until the sodium palmitate completely dissolved. The jar was removed from the bath and placed in a second 80° C.-controlled water bath (VWR 7×7 Stir PRO w/Temp probe). The first lid was replaced with a second lid containing two, 8 mm holes: one hole was in the center set for the impeller shaft and one hole offset half way between the edge and the center of the jar set for adding the remaining ingredients. A 4-blade impeller was installed by passing the shaft through the center hole in the lid and placing the blade into the mixture when fasting the lid. The impeller was set to spin at 450 rpm (Caframo BDC 3030). Finally, the oil/petrolatum blend (A9) or (A10) was added through the same hole. The impeller speed was increased to 750 rpm for two additional minutes. The final mixture was poured into 60 ml cups (Flak-Tech, Max 60 Cup Translucent Reorder Number: 501 222t) to cool and crystallize.

TABLE 25

| | |
|---|---|
| Sample CI | Sample CJ |
| Inventive | Comparative |

| | | | | |
|---|---|---|---|---|
| (3) Water | 69.54 | g | 71.52 | g |
| (A8) Laponite solution | 10.06 | g | 10.03 | g |

| % Suspension Agent | 0.52% | 0.50% | |
|---|---|---|---|
| (21) NaCl | — | 3.51 | g |

| | | | | |
|---|---|---|---|---|
| (8) NaP | 5.02 | g | 5.01 | g |

| | | |
|---|---|---|
| NaP wt % | 5% | 5% |

| | | | | |
|---|---|---|---|---|
| (A9) Oil/Petrolatum Blend | 18.028 | g | 10.041 | g |

| | | | |
|---|---|---|---|
| (A10) Oil/Petrolatum Blend | — | 10.09 | g |

| | | |
|---|---|---|
| Stability | 2 | 0 |
| Firmness | — | — |
| Temperature | — | — |
| Work | — | — |

### Example 11

This example demonstrates that it is possible to create stable, commercially viable compositions with a large weight amount of a very complex mixture of insoluble active agents on the order of 25 wt %, even at somewhat higher levels of suspension agents. It is believed that higher level of insoluble active (% IA)—such as petrolatum and insoluble oil, allow consumers to better recognize sensory experiences such as ‘feel’ and ‘smell’ of the compositions, when applied to skin. Both petrolatum and the insoluble oil will separate from the water during the formation without the use of the suspension agent. Not wishing to be bound by theory, it is believed that the suspension agent increases the viscosity of the compositions during preparation (e.g. Example 1), preventing separation of the insoluble active and requiring even higher levels of suspension agents. Example 1 demonstrates that a minimal level of suspension agent is needed for the suspension of the insoluble active, including only x-gum provided the levels are sufficiently high. Example 2 demonstrates that increasing the level of suspension agent can significantly soften the composition—some not crystallizing at all, and requiring additional crystallizing agent and salt. This example demonstrates that one can utilize up to 0.30 wt % x-gum to create compositions with 25 wt % insoluble active that meet the desired criteria of stability, thermal stability, firmness and water expression.

### (A11) Preparation of X-Gum in Glycerol Stock

The x-gum stock was prepared by adding 36.024 grams of glycerol (9) to 60 ml Speed Mixer Cup (Flak-Tech, Max 60 Cup Translucent Reorder Number: 501 222t). 4.015 grams of x-gum (5) were added to the cup. It was placed in the Speed Mixer (Flacktek, Inc.) and run at 3500 rpm for one minute. The mixture was allowed to sit quiescently for an hour at which point is was re-mixed at 3500 rpm for another 10 seconds.

### Preparation of Compositions

Part 1: Oil/Petrolatum Mixture: the oil mixture (A3) is added to a glass vial and placed in a heat block set to 60° C. The petrolatum (22) is heated until liquid, then added to the vial. The vial is agitated and held in heat block at 55° C. until use.

Part 2: Sample Preparation: Deionized water (3) was added to a 16 oz wide mouth glass jar (VWR).

All sodium chloride (21) was added to the jar for samples CK-CR; part of the sodium chloride (21) is added in example CS (first). The jar was swirled until the salt completely dissolved. It was then placed in a water bath (VWR 7×7 Stir PRO w/Temp probe) with the temperature controlled at 90° C. A magnetic stir bar was added to the mixture and set to turn at 200 rpm, creating a vortex in the mixture. Sodium palmitate (8) was added to the mixture. The jar was loosely capped to prevent water loss and to prevent pressurization. The mixture was stirred until the sodium palmitate completely dissolved. The jar was then removed from the first bath, and placed in a second controlled water bath (VWR 7×7 Stir PRO w/Temp probe) with the temperature controlled at 80° C. The first lid was replaced with a second lid, which contained two 8 mm holes: one hole centered for the impeller shaft and one hole offset half way between the edge and the center of the jar set for adding the remaining ingredients. A 4-blade impeller was installed by passing the shaft through the center hole in the lid and placing the blade into the mixture when fastening the lid. The impeller was spun at 500 rpm (Caframo BDC 3030). The xanthan gum stock solution (A11) was slowly added through the second hole using a syringe. Finally, the Oil/Petrolatum Mixture (Part 1) and preservative (1) was added through the same hole for samples CK-CR; the Oil/Petrolatum Mixture (Part 1), preservative (1) and balance of the sodium chloride (21) was added through the same hole for sample CS (second). The impeller speed was increased to 1,000 rpm for two additional minutes. The final mixture was poured into 60 ml cups (Flak-Tech, Max 60 Cup Translucent Reorder Number: 501 222t) to cool and crystallize. The solution was then divided into three 60 g plastic jars (Flak-Tech, Max 60 Cup Translucent, Cat #501 222t): one jar was filled to 50 ml and two jars filled to 25 ml. The samples were kept at 60° C. for one hour and then cooled at room temperature (25±3° C.) until solid. Firmness measurements were made on the 50 ml sample with the FIRMNESS TEST METHOD and a thermal stability measurement was made by the THERMAL STABILITY TEST METHOD on the 50 ml sample. Water-expression measurements were made by the WATER-EXPRESSION TEST METHOD on the two 25 ml samples.

TABLE 26

| | | | |
|---|---|---|---|
| Sample CK | Sample CL | Sample CM | Sample CN |
| Inventive | Inventive | Inventive | Inventive |

Part 1: Mixture

| | | | | | | | | |
|---|---|---|---|---|---|---|---|---|
| (A3) Oil | 36.06 | g | 27.059 | g | 45.22 | g | 44.99 | g |
| (22) Petrolatum | 36.07 | g | 27.01 | g | 45.13 | g | 15.31 | g |

| | | | | |
|---|---|---|---|---|
| % IAA | 24.0% | 18.0% | 30.0% | 20.0% |

Part 2: Sample

| (3) Water | 193.308 | g | 211.310 | g | 175.210 | g | 205.337 | g |
|---|---|---|---|---|---|---|---|---|
| (21) NaCl | 10.531 | g | 10.523 | g | 10.503 | g | 10.511 | g |
| (8) NaP | 14.99 | g | 14.99 | g | 15.062 | g | 15.01 | g |
| NaP wt % | 4.99% | | 4.99% | | 5.01% | | 5.00% | |
| (A10) x-gum stock | 9.03 | g | 9.02 | g | 9.37 | g | 9.05 | g |
| % Suspension Agent | 0.30% | | 0.30% | | 0.31% | | 0.30% | |
| (1) Euxyl PE 9010 | 0.30 | g | 0.31 | g | 0.32 | g | 0.32 | g |
| Stability | 2 | | 2 | | 2 | | 2 | |
| Firmness | 9.72N | | 12.36N | | 7.19N | | 8.13N | |
| Temperature | 51.7° | C. | 50.7° | C. | 54.4° | C. | 50.6° | C. |
| Work | 5,620 | J m−3 | 4,245 | J m−3 | 4,112 | J m−3 | 4,965 | J m−3 |

TABLE 27

| | | | |
|---|---|---|---|
| Sample CO | Sample CP | Sample CQ | Sample CR |
| Inventive | Inventive | Inventive | Inventive |

Part 1: Mixture

| | | | | | | | | |
|---|---|---|---|---|---|---|---|---|
| (A3) Oil | 45.20 | g | 45.13 | g | 27.05 | g | 35.99 | g |
| (22) Petrolatum | 30.21 | g | 23.99 | g | 15.12 | g | 15.25 | g |

| | | | | |
|---|---|---|---|---|
| % IAA | 25.0% | 23.0% | 14.0% | 17.1% |

Part 2: Sample

| (3) Water | 190.301 | g | 196.471 | g | 223.328 | g | 214.218 | g |
|---|---|---|---|---|---|---|---|---|
| (21) NaCl | 10.531 | g | 10.523 | g | 10.54 | g | 10.501 | g |
| (8) NaP | 15.15 | g | 15.02 | g | 14.99 | g | 15.03 | g |
| NaP wt % | 5.05% | | 5.01% | | 5.00% | | 5.01% | |
| (A10) x-gum stock | 9.21 | g | 9.13 | g | 9.12 | g | 9.116 | g |
| % Suspension Agent | 0.31% | | 0.30% | | 0.30% | | 0.30% | |
| (1) Euxyl PE 9010 | 0.330 | g | 0.308 | g | 0.312 | g | 0.312 | g |
| Stability | 2 | | 2 | | 2 | | 2 | |
| Firmness | 9.15N | | 9.32N | | 13.15N | | 12.54N | |
| Temperature | 51.3° | C. | 51.8° | C. | 49.7° | C. | 50.2° | C. |
| Work | 4,375 | J m−3 | 6,005 J m−3 | | 3,405 | J m−3 | 3,820 | J m−3 |

TABLE 28

Sample CS
Inventive

Part 1: Mixture

| | | |
|---|---|---|
| (A3) Oil | 45.20 | g |
| (22) Petrolatum | 30.21 | g |

| | |
|---|---|
| % IAA | 24.1% |

Part 2: Sample

| (3) Water | 200.785 | g |
|---|---|---|
| (21) NaCl (first) | 9.045 | g |
| (8) NaP | 15.01 | g |
| NaP wt % | 5.00% | |
| (A10) x-gum stock | 9.13 | g |
| % Suspension Agent | 0.30% | |
| (1) Euxyl PE 9010 | 0.297 | g |
| (21) NaCl (second) | 3.015 | g |
| Stability | 2 | |
| Firmness | 7.59N | |
| Temperature | 52.6° | C. |
| Work | 4,965 | J m−3 |

### Example 12

This example demonstrates a method of preparing a rheological solid personal care composition. A 5-kg batch of rheological solid personal care composition was prepared according to the following procedure:

First, water, NaCl, and NaOH were added to a main mixing vessel (2-gallon Ross mixer with planetary and high shear mixing elements). Heating and mixing of the main mixing vessel were initiated to provide an aqueous phase. Once the main mixing vessel reached 70±5° C., palmitic acid as an emulsifier was added to the main mixing vessel and mixed for approximately 10 minutes to ensure neutralization to sodium palmitate. The main mixing vessel continued to be heated to 80±5° C. Then, phenoxyethanol as a preservative and NaCl to improve thermal stability of the final rheological solid personal care composition were added to the main mixing vessel.

Xanthan gum and glycerin were added to a first pre-mix vessel (stainless steel container with overhead mixer fitted with pitch blade mixing element) and mixed to ensure that the xanthan gum was dispersed within the glycerin. This suspension agent pre-mix was then added to the main mixing vessel to add structure for dispersion of hydrophobic ingredients.

Petrolatum and fragrance were added to a second pre-mix vessel (stainless steel container with overhead mixer fitted with pitch blade mixing element) and heated to 40±5° C. while mixing to form a petrolatum-fragrance pre-mix. The petrolatum-fragrance pre-mix can comprise an insoluble active, preferably a topical drug active selected from the group of: menthol, nutmeg, camphor, eucalyptus, cedar leaf, thymol, and any combinations thereof.

The main mixing vessel was cooled to 65±5° C. and the petrolatum-fragrance pre-mix was added to the main mixing vessel. Sodium lactate as a hygroscopic component was further added to the main mixing vessel to stabilize the final crystalline structure of the rheological solid personal care composition. The main mixing vessel was then mixed for approximately 10 minutes. The cooling causes the sodium palmitate to crystallize, thereby enclosing the hydrophilic and hydrophobic components.

A rheological solid personal care composition manufactured by said process may comprise the following components:

TABLE 29

| Ingredient Name | Ingredient Function | % w/w |
|---|---|---|
| Water | Solvent | 61.52 |
| Sodium Lactate | Hygroscopic Stabilizer | 3.33 |
| Sodium Chloride | Temperature Stabilizer | 3.00 |
| Sodium Hydroxide | Base | 1.44 |
| Palmitic Acid | Palmitate Precursor/Emulsifier | 4.61 |
| Glycerin | Dispersant for X-gum | 2.70 |
| Xanthan Gum | Structurant | 0.30 |
| Petrolatum | Stabilizer | 8.00 |
| Fragrance | Fragrance | 15.00 |
| Phenoxyethanol | Preservative | 0.10 |

Not wishing to be bound by theory, it is believed that the basic unit operations described in the 5-kg process can be scaled with the size of the batch. Therefore, it is expected that the same making process described in the 5-kg process apply to commercial scale batches such as 1,000 kg using specific mix tanks. Further, while described as a batch process, it is expected such compositions may be prepared also in a continuous process.

It further follows that the order of addition of components into the 5-kg batch process is non-limiting. Laboratory scale batches show that order of addition can be adjusted (e.g. salt addition in examples CR and CS). It is believed that the order of addition can also be adjusted in commercial scale making processes.

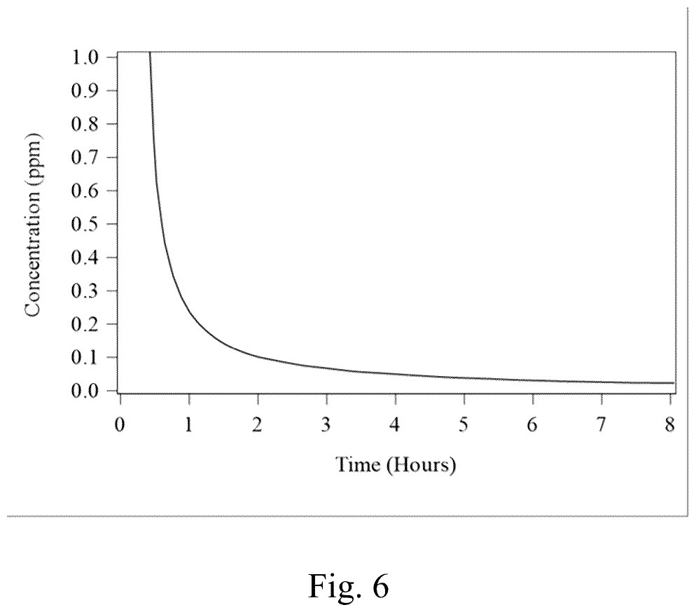

The release of fragrance compounds from the rheological solid personal care composition of Table 29 is evaluated using Selected Ion Flow Tube Mass Spectrometry (SIFT-MS). The concentration profile describes a consumer experience that includes a “burst” or strong release of fragrance upon application, followed by at least 15 minutes of fragrance release at a concentration above the odor detection threshold for a given fragrance. These data are illustrated in the plot of Concentration (ppm) of fragrance compounds vs. Time (Hours) provided in FIG. 6.

### Combinations

A. A rheological solid personal care composition comprising: (a) a crystallizing agent; (b) a suspension agent; (c) an insoluble active; and (d) an aqueous phase.

B. The rheological solid personal care composition according to Paragraph A, wherein the crystallizing agent is present in an amount from 0.01% to 10 wt. %, by weight of the rheological solid personal care composition, preferably from 0.1% to about 7 wt. %, more preferably from 1% to about 7%.

C. The rheological solid personal care composition according to Paragraph A or B, comprising from 0.01 to 2 wt. % of a suspension agent, by weight of the rheological solid personal care composition, preferably 0.05 to 1 wt. %, more preferably from 0.1 to 0.5 wt. %.

D. The rheological solid personal care composition according to any of the preceding paragraphs, comprising from 0.1 to 30 wt. % of an insoluble active, by weight of the rheological solid personal care composition, preferably from 0.1 to 25 wt. %, more preferably from 0.5 to 15 wt. %.

E. The rheological solid personal care composition according to any of the preceding paragraphs, wherein the crystallizing agent comprises a salt of fatty acids containing from about 12 to about 20 carbon atoms.

F. The rheological solid personal care composition according to any of the preceding paragraphs, wherein the crystallizing agent is a metal salt.

G. The rheological solid personal care composition according to Paragraph F, wherein the metal salt is at least one of sodium stearate, sodium palmitate, and sodium myristate.

H. The rheological solid personal care composition according to any of the preceding paragraphs, wherein the insoluble active is an insoluble active particle comprising an insoluble oil.

I. The rheological solid personal care composition according to Paragraph H, wherein the insoluble active particle further comprises a hydrophobic non-aqueous vehicle.

J. The rheological solid personal care composition according to Paragraph I, wherein the rheological solid personal care composition comprises from about 1 to about 15% of the hydrophobic non-aqueous vehicle, by weight of the rheological solid personal care composition, preferably from 3 to 12 wt. %, more preferably from 5 to 10 wt. %.

K. The rheological solid personal care composition according to Paragraph H, wherein the rheological solid personal care composition comprises from about 4 to about 10 wt. % of the insoluble oil.

L. The rheological solid personal care composition according to any of the preceding paragraphs, wherein the suspension agent comprises a polysaccharide.

M. The rheological solid personal care composition according to any of the preceding paragraphs, wherein the suspension agent comprises a first polysaccharide and a second polysaccharide, wherein the first polysaccharide is xanthan gum and the second polysaccharide is selected from the group consisting of: konjac gum, locust bean gum, and combinations thereof.

N. The rheological solid personal care composition according to any of the preceding paragraphs, having a stability grade of 1 or greater as determined by the PHASE STABILITY TEST METHOD.

O. The rheological solid personal care composition according to any of the preceding paragraphs, having a thermal stability greater than about 30° C. as determined by the THERMAL STABILITY TEST METHOD.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Values disclosed herein as ends of ranges are not to be understood as being strictly limited to the exact numerical values recited. Instead, unless otherwise specified, each numerical range is intended to mean both the recited values and any real numbers including integers within the range. For example, a range disclosed as “1 to 10” is intended to mean “1, 2, 3, 4, 5, 6, 7, 8, 9, and 10” and a range disclosed as “1 to 2” is intended to mean “1.1, 1.2, 1.3, 1.4, 1.5, 1.6, 1.7, 1.8, 1.9, and 2.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A rheological solid personal care composition comprising:
a. a crystallizing agent,
wherein the crystallizing agent comprises a metal salt selected from the group consisting of sodium stearate, sodium palmitate, sodium myristate, and mixtures thereof, and
wherein the crystallizing agent is present in an amount of from about 0.01% to about 10%, by weight of the rheological solid personal care composition;
b. a suspension agent;
c. an insoluble active; and
d. an aqueous phase;
wherein the rheological solid personal care composition comprises a crystalline mesh, wherein the crystalline mesh comprises crystalline particles formed from the crystallizing agent.

2. The rheological solid personal care composition of claim 1, wherein the rheological solid personal care composition is characterized by at least one of the following characteristics:
a. a firmness between 0.1 N and 50.0 N;
b. having a thermal stability greater than about 30° C.;
c. having a liquid expression of between about 100 J m-3 and about 6000 J m-3;
d. having a stability grade of 1 or greater;
e. having a stability grade of 2 or greater.

3. The rheological solid personal care composition of claim 1, wherein the crystallizing agent is present in an amount of from about 1% to about 7%, by weight of the rheological solid personal care composition.

4. The rheological solid personal care composition of claim 1, wherein the suspension agent comprises a polysaccharide.

5. The rheological solid personal care composition of claim 4, wherein the polysaccharide comprises xanthan gum.

6. The rheological solid personal care composition of claim 4, wherein the total concentration of polysaccharide present in the rheological solid personal care composition is from about 0.01 to about 1.0 wt. %, by weight of the rheological solid personal care composition.

7. The rheological solid personal care composition of claim 1, wherein the insoluble active comprises a topical drug active selected from the group consisting of menthol, nutmeg, camphor, eucalyptus, cedar leaf, thymol, and mixtures thereof.

8. The rheological solid personal care composition of claim 1, wherein the insoluble active comprises an insoluble active particle comprising an insoluble oil.

9. The rheological solid personal care composition of claim 8, wherein the rheological solid personal care composition comprises from about 4% to about 15%, by weight of the composition, of the insoluble oil.

10. The rheological solid personal care composition of claim 1, wherein the rheological solid personal care composition further comprises a hydrophobic non-aqueous vehicle.

11. The rheological solid personal care composition of claim 10, wherein the hydrophobic non-aqueous vehicle comprises a material selected from the group consisting of liquid petrolatum, petrolatum, mineral oil, glycerin, synthetic oils, fats, silicone, silicone derivatives, polyvinylacetate, natural wax, synthetic wax, hydrocarbons, hydrocarbon derivatives, vegetable oil waxes, vegetable oils, pentaerythrityl tetraisostearate, and a combination thereof.

12. The rheological solid personal care composition of claim 10, wherein the hydrophobic non-aqueous vehicle comprises a material selected from the group consisting of liquid petrolatum, petrolatum, mineral oil, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, fish oil, oleic acid, and mixtures thereof.

13. The rheological solid personal care composition of claim 10, wherein the hydrophobic non-aqueous vehicle comprises liquid petrolatum, petrolatum, or a mixture thereof.

14. The rheological solid personal care composition of claim 10, wherein the rheological solid personal care composition comprises from about 1 to about 15%, by weight of the rheological solid personal care composition, of the hydrophobic non-aqueous vehicle.

15. The rheological solid personal care composition of claim 1, wherein the crystalline particles of the crystalline mesh comprise fiber-like crystalline particles, wherein the fiber-like crystalline particles are characterized by having a length in a direction of its longest axis being greater than ten times a length of the particle in any orthogonal direction.

16. The rheological solid personal care composition of claim 1, wherein the rheological solid personal care composition is in the form of a stick intended for topical application to skin.

17. A rheological solid personal care composition comprising:
a. a crystallizing agent, wherein the crystallizing agent comprises sodium palmitate;
b. a suspension agent, wherein the suspension agent comprises xanthan gum;
c. an insoluble active;
d. petrolatum;
e. water;
f. sodium chloride;
g. glycerin; and
h a preservative;
wherein the rheological solid personal care composition comprises a crystalline mesh, and wherein the rheological solid personal care composition is in the form of a stick intended for topical application to skin.

18. The rheological solid personal care composition according to claim 17, wherein the rheological solid personal care composition is characterized by at least one of the following characteristics (a. through e.):
a. a firmness between 0.1 N and 50.0 N;
b. having a thermal stability greater than about 30° C.;
c. having a liquid expression of between about 100 J m-3 and about 6000 J m-3;
d. having a stability grade of 1 or greater;
e. having a stability grade of 2 or greater.

19. The rheological solid personal care composition according to claim 18, wherein the rheological solid personal care composition is characterized by at least two of the characteristics (a. through e.).

20. A method of treating nasal congestion, common cold, flu, cough, dry cough, chest congestion, or muscle aches and pains, the method comprising the step of contacting a body of the user with the rheological solid personal care composition of claim 1.
